(19) United States
(12) Patent Application Publication
(10) Pub. No.: US 2024/0226880 A1
(43) Pub. Date: Jul. 11, 2024

(54) MICROFLUIDIC CHIP, SYSTEM, AND METHOD FOR DETERMINING CELL DEFORMABILITY

(71) Applicant: City University of Hong Kong, Hong Kong (CN)

(72) Inventors: Bee Luan KHOO, Hong Kong (CN); Haojun HUA, Hong Kong (CN); Shangjie ZOU, Hong Kong (CN)

(21) Appl. No.: 18/150,852

(22) Filed: Jan. 6, 2023

(51) Int. Cl. B01L 3/00

(52) U.S. Cl. CPC B01L 3/502715; B01L 3/50273; B01L 3/502761; B01L 2300/0816; B01L 2300/0864; B01L 2400/0487

(57) ABSTRACT

One embodiment includes a microfluidic chip for implementing cell deformability. The microfluidic chip comprises an inlet configured to receive cells, an outlet configured to output the cells, a plurality of main channels, and one or more bypass channels. The main channels are disposed between the inlet and the outlet and are provided with microconstrictions that are parallelized such that images of the cells are captured within a single field of view (FOV) when the cells pass through the microconstrictions and are deformed therein. The one or more bypass channels are independent from the main channels, thereby stabilizing pressure drop between the inlet and the outlet.

### FIELD OF THE INVENTION

The present invention relates generally to cell deformability.

### BACKGROUND

Deformability of cells, which indicates cells' capability to deform under external forces, is an emerging biomarker in industries, such as medical industry, such as clinic applications, due to its correlations with cells' physiological statuses. For example, as a property directly related to structural features like cytoskeleton components, the density of nucleus, and chromatin texture, cells' deformability can reflect cells' physiological statuses. Therefore, cell deformability can be a useful parameter in medical applications. For example, it can be applied to detection of infections of malaria, sepsis, bacterial infection, and evaluation of the differentiation of stem cells, pathological conditions of cardiac cells, or evaluation of metastatic potentials of cancer cells. For example, deformability of cells is highly linked to pathological conditions of many diseases. However, this valuable mechanical property has been poorly utilized in industries, such as when it comes to actual drug discovery or clinical applications.

Currently, representative standard methods for deformability measurement include atomic force microscopy, micro-aspiration, optical stretching, parallel-plate rheology, and so on. Common problems for these conventional methods include low throughput (e.g., <10 cells per minute). Apart from that, these conventional techniques for deformability measurement usually unavoidably miss some important information. For example, shear modulus is important for evaluating cells' ability for migration and retention, which is important in clinical applications. However, conventional methods can at most poorly measure this property.

Recent advances in measuring cells' deformability have been mainly contributed by microfluidics. The most high-throughput phenotype of microfluidics-based deformability cytometry is extensional flow deformability cytometry (xDC). xDC methods, when applied, have achieved a throughput of >1000 cells per second. Another phenotype of microfluidic deformability cytometry is shear flow deformability cytometry (sDC). The throughput of sDC is normally >100 cells per second. Although these technologies can achieve relatively high-throughput, they require high expense imaging equipment and sophisticated flow control, which makes them difficult for being widely applied in actual application scenarios. In comparison to sDC and xDC, constriction-based deformability cytometry (cDC) has the lowest throughput. However, only cDC among the three methods has the ability to evaluate cells' migration and retention, due to the direct contact of cells with channel walls during assay running. Moreover, xDC and sDC usually require very high frame rates (>2000 fps) and expensive high-speed cameras, while cameras with <1000 fps are already applicable for cDC.

New apparatus or devices, systems, and methods that assist in advancing technological needs and industrial applications in relation to cell deformability are desirable.

### SUMMARY

One embodiment includes a microfluidic chip for implementing cell deformability. The microfluidic chip comprises an inlet configured to receive cells, an outlet configured to output the cells, a plurality of main channels, and one or more bypass channels. The plurality of main channels are disposed between the inlet and the outlet and are provided with microconstrictions that are parallelized such that images of the cells are captured within a single field of view (FOV) when the cells pass through the microconstrictions and are deformed therein. The one or more bypass channels are disposed between the inlet and the outlet and independent from the plurality of main channels, thereby stabilizing pressure drop between the inlet and the outlet.

Other example embodiments are discussed herein.

### BRIEF DESCRIPTION OF THE DRAWINGS

FIG. 1 illustrates a microfluidic chip in accordance with certain embodiments.

FIG. 2A illustrates a microfluidic chip in accordance with certain embodiments.

FIG. 2B illustrates velocity distribution in a microfluidic chip derived from Laminar flow velocity simulation under 50 μL/min flow rate using a software COMSOL in accordance with certain embodiments.

FIG. 2C illustrates configuration of microconstrictions of FIG. 2B that are divided into four groups with each group including nine microconstrictions.

FIG. 2D shows velocity distribution along moving direction of cells in a certain region including a single microconstriction for the microfluidic chip of FIG. 2B.

FIG. 3A illustrates a process of a cell travelling through a microconstriction in accordance with certain embodiments.

FIG. 3B illustrates the cell of FIG. 3A fully within the microconstriction and deformed therein.

FIG. 4A illustrates a cell deformed in a microconstriction in accordance with certain embodiments, where Re is half of the width of the microconstriction.

FIG. 4B illustrates a process of the cell of FIG. 4A travelling through the microconstriction.

FIG. 5 illustrates a system for determining cell deformability in accordance with certain embodiments.

FIG. 6 illustrates a system for determining cell deformability in accordance with certain embodiments.

FIG. 7 is a diagram illustrating algorithms of a computational framework implemented in a system for determining cell deformability in accordance with certain embodiments.

FIG. 8 illustrates a structure of YOLOv5 in accordance with certain embodiments.

FIG. 9A illustrates a graphical principle of background subtraction method (upper panel) and an overview of training set creation for YOLOv5 (lower panel) in accordance with certain embodiments.

FIG. 9B illustrates images in the training set after being processed for mosaic augmentation.

FIG. 9C illustrates training loss, precision, recall and mean average precision (mAP) of YOLOv5.

FIG. 9D illustrates detection of cells by established or trained YOLOv5 model in accordance with certain embodiments.

FIG. 10 illustrates a structure of Deep SORT in accordance with certain embodiments.

FIG. 11A shows a brief demonstration of cell tracking based on Deep SORT in accordance with certain embodiments.

FIG. 11B illustrates two types of cells with different deformability that are differentiated by passage time in accordance with certain embodiments.

FIG. 11C illustrates application of focusing areas for passage time quantification in accordance with certain embodiments.

FIG. 11D shows images illustrating how a cell is tracked in focusing areas in accordance with certain embodiments.

FIG. 12A is a diagram showing blocks unit used in Unet in accordance with certain embodiments.

FIG. 12B is a diagram showing residual unit with identity mapping used in ResUnet in accordance with certain embodiments.

FIG. 12C is a diagram showing an architecture of ResUnet++ in accordance with certain embodiments.

FIG. 12D shows segmentation of a cell from its original image based on the trained ResUnet++ model with the architecture of FIG. 12C in accordance with certain embodiments.

FIG. 13A shows passage time for MCF-7 and MDA-MB-231 in accordance with certain embodiments. Statistical significance is calculated by two-tailed Student's t-test (P-value <0.05: *; <0.01: **; <0.001: ***; <0.0001: ****).

FIG. 13B shows passage time receiver operating characteristic (ROC) curve of MCF-7 and MDA-MB-231 in accordance with certain embodiments.

FIG. 13C shows deformation index for MCF-7 and MDA-MB-231 in accordance with certain embodiments.

FIG. 13D shows the deformation index ROC curve of MCF-7 and MDA-MB-231 in accordance with certain embodiments.

FIG. 13E shows cell diameters for cancer cells and white blood cells (WBCs) in accordance with certain embodiments.

FIG. 13F shows cell diameter ROC curve of cancer cells and WBCs in accordance with certain embodiments.

FIG. 13G shows cell diameter for MCF-7 and MDA-MB-231 in accordance with certain embodiments.

FIG. 13H shows cell diameter ROC curve of MCF-7 and MDA-MB-231231 in accordance with certain embodiments.

FIG. 14A shows Youden's J Statistic for optimal thresholds of passage time for differentiating MCF-7 and MDA-MB-231 in accordance with certain embodiments.

FIG. 14B shows Youden's J Statistic for optimal thresholds of deformation index for differentiating MCF-7 and MDA-MB-231 in accordance with certain embodiments.

FIG. 15A are images showing deformation of cells after entering microconstriction and then passes through the microconstriction in accordance with certain embodiments.

FIG. 15B illustrates trajectory of MCF-7 cells in the form of position vs time curve when traveling through a microconstriction in accordance with certain embodiments. A schematic diagram of microconstriction is shown in the line plot to demonstrate the correspondent relations between the position value on x-axis and the actual position in a microconstriction.

FIG. 15C shows average time vs position curve of MCF-7 cells through a microconstriction, where the time includes creep time and transit time, in accordance with certain embodiments.

FIG. 15D illustrates trajectory of MDA-MB-231 cells in the form of position vs time curve when traveling through a microconstriction in accordance with certain embodiments.

FIG. 15E shows average time vs position curves of MDA-MB-231 cells through a microconstriction in accordance with certain embodiments.

FIG. 16A illustrates passage time (in log 10 scale) vs cell size (represented by cell's area) for MCF-7 and MDA-MB-231 cells, where data points in black and grey represent MCF-7 and MDA-MB-231 cells respectively, and dash lines in black and grey for MCF-7 and MDA-MB-231 cells respectively are theoretical cell size vs t_{creep} curves calculated by Equation (5) using the calculated cl indexes, in accordance with certain embodiments.

FIG. 16B illustrates passage time (in log 10 scale) vs cell size (represented by cell's area) for MCF-7 and MDA-MB-231 cells, where data points in black and grey represent MCF-7 and MDA-MB-231 cells respectively according to their local density, and dash lines in black and grey for MCF-7 and MDA-MB-231 cells respectively are theoretical cell size vs t_{creep} curves calculated by Equation (5) using the calculated cl indexes, in accordance with certain embodiments.

FIG. 16C are Kernel density estimation plots showing distribution of cl indexes for MCF-7 (represented by C), MDA-MB-231 (represented by D), and mixed group A (represented by A), and mixed group B (represented by B) in accordance with certain embodiments.

FIG. 16D shows values of cl index for MCF-7, MDA-MB-231, the mixed group A and the mixed group B of FIG. 16C respectively, where statistical significance is calculated by two-tailed Student's t-test (P-value >0.05: ns; 0.05: *; <0.01: **; <0.001: ***<0.0001: ****).

FIG. 16E shows classification result by using support vector machine (SVM) for MCF-7 and MDA-MB-231 in accordance with certain embodiments.

### DETAILED DESCRIPTION

Example embodiments relate to microfluidic chip, system, and method for determining cell deformability.

Deformability of cells is a useful indicator in many industries. For example, cell deformability is related to the pathology of many diseases. However, the difficulty in measuring cells' deformability has long been an obstacle. Many existing apparatus, systems, or methods for determining cell deformability have various disadvantages. For example, many existing technologies for deformability measurement (e.g. atomic force microscopy, micro-aspiration, optical tweezer, etc.) focus on precise measurement of Young's modulus. These techniques require one-by-one measurement and involve expensive equipment and sophisticated operations, therefore are unsuitable for industrial applications, such as clinical applications.

Those existing technologies that employ microfluidic models for deformability cytometry, such as sDC, xDC, and cDC, use different methods to induce deformation. These techniques are unfavorable in various aspects, such as still relatively low throughput, complicated operations, high cost, low sensitivity, low accuracy, difficult to manufacture, unsuitable for mass production, etc., which has significantly limited their industrial applications.

Example embodiments solve one or more of those problems associated with the existing technologies and provide technical solutions including microfluidic chip, system, or method for determining cell deformability that perform in novel ways and achieve various technical advantages.

Example embodiments include a microfluidic chip provided with parallelized microconstrictions, such that multiple cells can pass through the parallelized microconstrictions simultaneously and deformed therein. This arrangement of microconstrictions enables simultaneous observation of multiple cells passing through the microconstrictions in a single field of view (FOV) and avoids each time deformation of only one cell is measured, thereby significantly improving efficiencies and throughput.

Example embodiments include a microfluidic chip that includes an inlet and an outlet, a plurality of main channels disposed between the inlet and the outlet and provided with microconstrictions that have a parallelized configuration. The microfluidic chip further includes one or more bypass channels that stabilize pressure drop between the inlet and the outlet.

In some embodiments, a fluid sample including high concentration of cells is injected into the microfluidic chip. Some microconstrictions may be blocked or jammed by the cells. When this happens, the one or more bypass channels provide another route for some cells, thereby reducing resistance from the inlet to the outlet, releasing the blockage, and stabilizing the pressure within the microfluidic chip. This has an extra advantage of reducing errors of measurement, such as readouts. In the meanwhile, as a large number of cells are passing through and processed, having some of the cells flow into the bypass channels does not cause negative effects on final readout and evaluation.

In some embodiments, the microfluidic chip may be called as a highly parallelized constriction-based deformability cytometry (HPcDC). The term “HPcDC” itself, however, should not be understood as imposing any limitation to the microfluidic chip.

Example embodiments include a system for determining cell deformability. The system includes a microfluidic chip having parallelized microconstrictions. The system includes an image capturing device, such as a camera (such as a high-speed camera), to capture images (such as videos) of the cells when the cells travel through the microfluidic chip, particularly when travelling through the parallelized microconstrictions. The captured images include data related to morphological and motional parameters of the cells. A computing device communicates with the image capturing device and includes a computational framework. The computational framework is implemented with an artificial neutral network (ANN) having various machine learning tools, such as deep learning modes or algorithms, such as modes for cell detection, tracking, segmentation, and/or classification, etc. The computational framework automates generation of a training set for training various machine learning modes, and performs multi-object tracking, segmentation, and quantification of cells, such as identifying or determining cells' one or more properties. As such, manual generation of a training set for feeding to the ANN is avoided, thereby improving training efficiency, and in the meanwhile, expanding applications to a wider range of scenarios particularly when a large amount of training data is needed, which makes manual generation of training data undesirable or even impossible.

In some embodiments, the system is called an ATMQcD system or an ATMQcD platform. The term “ATMQCD” itself, however, should not be understood as imposing any limitation to the system.

In a system as described herein according to one or more embodiments, a fluid sample, such as urine, plasma, or blood, includes a plurality of cells or many cells and is delivered to a microfluidic chip having parallelized microconstrictions. The cells are deformed when passing through the microconstrictions. Morphological and motional parameters of the cells during deformation are captured or recorded by a camera and analyzed by a computational framework implemented in a computing device of the system. In some embodiments, the computational framework uses a background subtraction method or process and includes YOLOv5 algorithm or mode, Deep SORT algorithm or mode, and ResUnet++ algorithm or mode. In some embodiments, the precision of YOLOv5 detection results reaches as high as 0.901, and Passive-Aggressive (PA) algorithm and Intersection over Union (IoU) of ResUnet++ prediction results reach as high as 0.9859 and 0.9857 respectively. In some embodiments, the background subtraction method is implemented by OpenCV module of Python 3.8. YOLOv5, Deep SORT, and ResUnet++ are implemented by PyTorch. In some embodiments, YOLOv6 or YOLOv7 is used to replace YOLOv5, while Strong SORT is used to replace Deep SORT.

According to some embodiments, validation on two breast cancer cell lines (MCF-7 and MDA-MB-231) varied in invasiveness is conducted and demonstrates the system's good capability for cell classification. Quantification of passage time and size of cells based on the system according to one or more embodiments achieves a classification accuracy of 87.8% in distinguishing MCF-7 and MDA-MB-231, and 89.5% in distinguishing cancer cells from white blood cells (WBCs). Apart from comparing breast cancer cells with different metastatic potentials, the systems according to certain embodiments can also be applied to study difference in deformability between other contrasting cell types, like comparing T24 with UMUC3 (bladder cancer cells with different metastatic potentials), or comparing NCI-H2452 with MeT-5A (mesothelioma and healthy mesothelium cells).

Example embodiments include a system for determining cell deformability, where the system includes a robust and transferable computational framework for multi-object tracking and quantification on microfluidics, especially in the task of deformability measurement, which, for example, fosters development of high-throughput assays for clinical applications.

Example embodiments include one or more methods for determining cell deformability. The methods, for example, can be performed by using the system according to one or more embodiments as described herein.

Embodiments include one or more methods comprising delivering a fluid sample including cells into a microfluidic chip such that the cells flow through a plurality of main channels of the microfluidic chip and are deformed by a plurality of parallelized microconstrictions, recording images of the cells within a single field of view (FOV) by a camera when the cells travel through the parallelized microconstrictions, thereby generating recorded images, and determining deformation index for the cells by processing the recorded images by using a trained ANN.

By way of example, the methods automate generation of a training set by using a background subtraction method, and train an ANN using the training set, thereby producing the trained ANN. In some embodiments, to train the ANN, the methods train YOLOv5 using images and annotation derived from the recorded images by using the background subtraction method, track the cells using YOLOv5 and Deep SORT, segment the images derived from the recorded images when the cells crossing focusing areas to obtain segmented images, and train ResUnet++ using the segmented images.

By way of example, the methods detect the cells from the recorded images by using trained YOLOv5, track trajectory of the cells by using Deep SORT, and calculate deformation index and size of the cells by using ResUnet++. In some embodiments, the methods set focusing areas for determining entry time and leaving time for each of the cells when traveling through respective microconstriction, and calculate passage time for each of the cells by using the trained YOLOv5 and the Deep SORT. In some embodiments, the methods categorize different cell types based on passage time and size of the cells by using a support vector machine algorithm.

According to certain embodiments, background subtraction method is employed to determine each cell's position. The images and annotation derived from the background subtraction method are used to train YOLOv5. Cell tracking is conducted based on conjugating YOLOv5 and Deep SORT. By way of example, YOLOv5 and Deep SORT are combined for purpose of cell tracking. The results of YOLOv5 can be used as the input of Deep SORT. YOLOv5 can output each cells' position (such as coordinates X, Y) and size (such as width W, and height H). Deep SORT can then use each cells' position from YOLOv5 to generate and predict movement for each cell, thereby obtaining cell speed and trajectories. Passage time for cells can be calculated by using YOLOv5 and Deep SORT. Focusing areas or focusing points or focal points can be set manually or automatically. For example, the focusing point's coordinates (X1, Y1) can be manually entered in order to optimize the output. For another example, the focusing point's coordinates can be set automatically by using machine leaning algorithms.

According to some embodiments, cell images are cut or divided (such as being cut into size of pixel: 200×200 or 50×50) by algorithms when the cells are crossing the focusing areas to obtain cut images. ResUnet++ is then trained using the cut images as input. For example, image processing by the ResUnet++ algorithm may be influenced by the input picture size. If the algorithm uses the whole video picture or image (for example, an image including 900*540 pixels) as the input, this may result in low-quality segmentation results and also increase operation time of the algorithm. If the picture or image is cut or divided into small areas or regions or slices (e.g., 100*100 pixels for each slice) with the cell located substantially at the center when the cell passes through the respective focusing area, the algorithm's speed and precision are improved. Deformation index and size of the cell can be calculated by using ResUnet++, and the passage time and the area size of each cell can be connected. For example, each cell can be assigned a unique identity (ID). By associating the obtained passage time and cell size, etc. with the respective ID, the cell's attributes can be connected to the cell.

Once the ANN is trained, the trained ANN can be used to detect and tack cells, segment cell images, conduct quantification of cell parameters of interest, such as passage time, deformability index, cell size, etc. The methods are capable of multi-cell tracking with improved efficiency and accuracy. By way of example, cell images can be cut before inputting to the trained ResUnet++ for further processing to determine deformation index and cell size.

The systems and/or methods as described herein according to certain embodiments achieve improved efficiency in measuring deformability of cells, and solve problems present in existing deformability cytometric systems in terms of processing throughput and costs, etc., without sacrificing sensitivity.

The systems as described herein according to certain embodiments are easy to operate compared with existing systems. Operators can easily operate the systems after they have taken basic training. Many parts or units of the systems can automatically run without human intervention to produce results.

The systems as described herein according to certain embodiments can work under 50 μL/min flow rate of fluid sample and process up to 25,000 cells per minute, which significantly surpasses existing methods or systems.

The systems and methods as described herein according to certain embodiments have a wide range of applicability. They can be applied in context of scientific discovery and/or clinical detection. The systems are portable and can be easily installed in various sites, such as hospitals.

The systems and methods as described herein according to certain embodiments can deal with high dimensional information. For example, the systems can measure morphological and motional parameters related to deformability, thereby providing high-dimensional measurement on single cells and being suitable for applications that require precise classifications.

The systems as described herein according to certain embodiments have low cost. The systems do not require sophisticated equipment, expensive consumables, or expensive high-speed cameras (high-speed cameras can be applied but not essential). The cost for fabrication and operation is much lower than many existing technologies, such as sDC, xDC, etc.

The systems as described herein according to certain embodiments are easy to fabricate. The systems can be easily manufactured using photolithography and soft lithography. The fabrication is much easier and has lowered cost than many existing systems, such as electrical signal-based microfluidic deformability cytometry.

The systems as described herein according to certain embodiments have improved sensitivity. For example, clinical application requires evaluation of cell migration and retention. The system is more sensitive in evaluating cell migration and retention than many existing systems or methods, such as sDC and xDC.

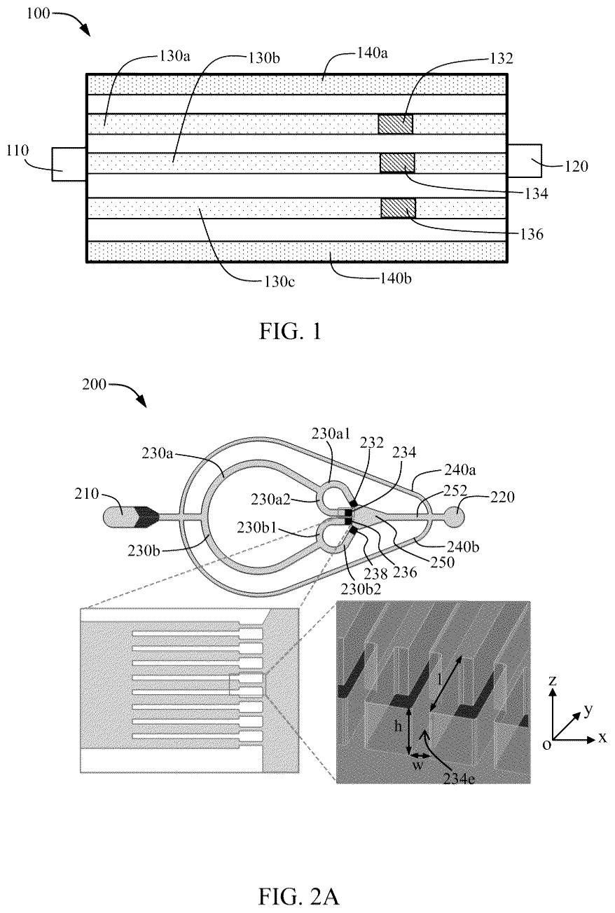

FIG. 1 illustrates a microfluidic chip in accordance with certain embodiments. The microfluidic chip can receive a fluid sample, such as urine, plasma, or blood. The fluid sample includes cells, such as tissue cells, stem cells, myeloid cells, or white blood cells. The cells are deformed within one or more certain regions in the microfluidic chip such that relevant data are collected and subsequently processed to determine deformability of cells.

As illustrated, the microfluidic chip 100 includes an inlet 110, an outlet 120, and a plurality of main channels 130a. 130b, 130c disposed between the inlet 110 and the outlet 120. Each main channel is provided with a microconstriction or a group of microconstrictions, and as a result, for illustrating purpose only, three microconstrictions 132, 134, 136 are shown. Each microconstriction includes one or more narrowed passages or channels such that a cell is deformed or squeezed or pressed along certain direction (such as perpendicular to the moving direction) when it travels through the narrowed passage.

The fluid sample generally includes many cells. When the fluid sample is delivered to the microfluidic chip 100 via the inlet 110, each cell will generally move toward the outlet 120 along a respective main channel. The cell will be deformed when it enters respective microconstriction. As the microconstrictions 132, 134, 136 are parallelized, multiple cells will be deformed simultaneously and their images can be captured within a single field of view (FOV), thereby realizing multi-object tracking and improved throughput.

The microfluidic chip 100 further includes one or more bypass channels 140a, 140b that are disposed between the inlet 110 and the outlet 120 and independent from the main channels 130a, 130b, and 130c. The bypass channels 140a, 140b do not include any microconstriction. The bypass channels 140a, 140b allow certain cells to pass through when there are many cells or when some microconstrictions are blocked or jammed, thereby reducing resistance and stabilizing pressure drop between the inlet 110 and the outlet 120.

As illustrated and optionally, the bypass channels 140a, 140b surround the main channels 130a, 130b, and 130c. This is advantageous as cells are expected to enter main channels and only in certain scenarios move toward the outlet via the bypass channels.

Three main channels, three microconstrictions and two bypass channels etc. are shown in FIG. 1. This is for only purpose of illustrating. The number of main channels or microconstrictions can be less than three or greater than three. The number of bypass channels can be one or greater than two. Each of the microconstrictions as illustrated may represent one or more microconstrictions, or one or more groups of microconstrictions with each group including one or more microconstrictions or one or more sub-groups of microconstrictions.

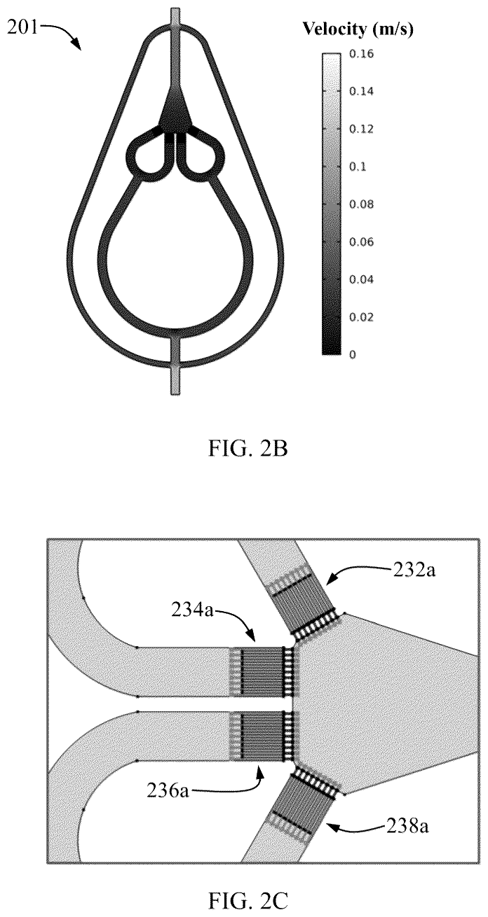

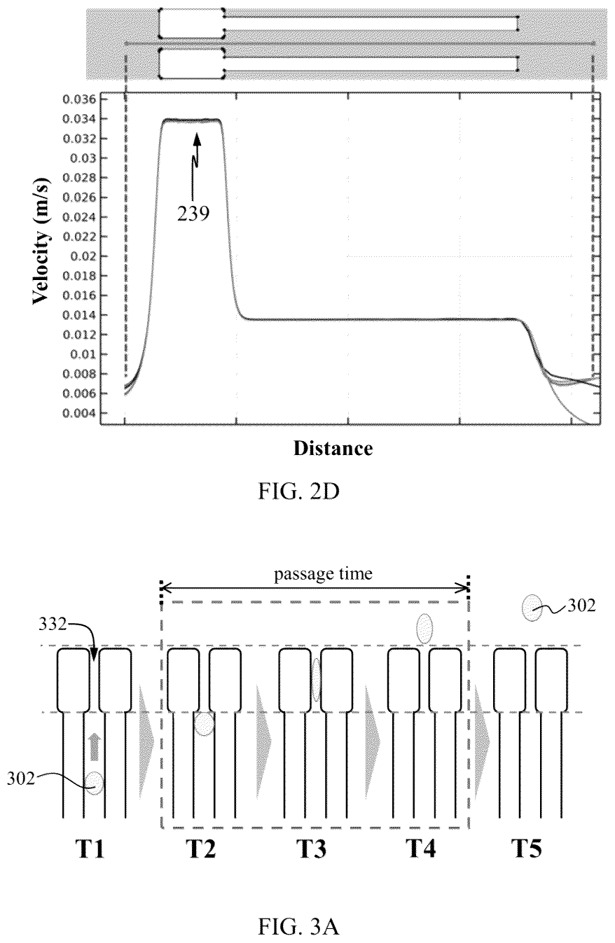

FIG. 2A illustrates a microfluidic chip in accordance with certain embodiments. The microfluidic chip 200, for example, can be a specific implementation of the microfluidic chip 100 with reference to FIG. 1.

The microfluidic chip 200 includes an inlet 210, an outlet 220, main channels 230a and 230b connecting the inlet 210 to the outlet 220, and bypass channels 240a and 240b disposed between the inlet 210 and the outlet 220 and independent from the main channels 230a and 230b.

Optionally and by way of example, the main channel 230a is branched into sub-channels 230a1 and 230a2, and the main channel 230b is branched into sub-channels 230b1 and 230b2. Each set of sub-channels may be considered as part of the respective main channel from which they are branched. The sub-channels 230a1, 230a2, 230b1, and 230b2 are provided with microconstrictions or groups of microconstrictions 232, 234, 236, and 238 respectively for deforming cells when the cells pass through the microconstrictions. The microconstrictions 232, 234, 236, and 238 are parallelized such that images of the cells are captured within a single FOV.

Optionally and by way of example, all the microconstrictions in a microfluidic chip can be categorized into m groups and the number of microconstrictions in each group is n, m and n being positive integers and greater than one. In the present embodiment as shown in FIG. 2A, m=4 and n=9. This is for illustrating purpose only. In some embodiments, the microconstrictions can be grouped into 2 or 3 groups, or more than 4 groups, such as 5, 8, 10, 16 groups, etc. In some embodiments, the number of microconstrictions in each group can be less than 9, such as 2, 4, 8, or greater than 9, such as 12, 16, 20, etc.

By way of example, the size or dimensions for all the microconstrictions can be same or different. The size or dimensions for some microconstrictions can be same but different from that for other microconstrictions. By way of example, each of the microconstrictions has a width (w) in a range from 9 μm to 11 μm, such as 9 μm, 10 μm, 11 μm, in a first direction (such as x direction), a height (h) in a range from 25 μm to 32 μm, such as 25 μm, 30 μm, 32 μm, in a second direction (such as z direction), and a length (l) in a range from 55 μm to 75 μm, such as 55 μm, 60 μm, 70 μm, 75 μm, in a third direction (such as y direction). FIG. 2A shows configuration of a representative microconstriction 234e with the three geometric dimensions.

Optionally and as illustrated, the output of the sub-channels 230a1, 230a2, 230b1, and 230b2 converge to a sink 250 that is connected to the outlet 220 through an elongated channel 252. In some embodiments, the output of the sub-channels, i.e., the ends or terminals opposite to the inlet 210, converge directly to the outlet 220.

Optionally and as illustrated, the bypass channels 240a and 240b surround the main channels 230a and 230b, the sub-channels 230a1, 230a2, 230b1, and 230b2, the microconstrictions 232, 234, 236, and 238, and the sink 250. Some embodiments may include only a single bypass channel, or three or more bypass channels.

The microfluidic chip can be manufactured using proper methods, such as photolithography and/or soft-lithography. In some embodiments, the microfluidic chip is fabricated as below. A microchannel layer is made by standard photolithography and soft-lithography based on negative photoresist (Cat #SU-8 2025, MicroChem) and polydimethylsiloxane (PDMS) (Cat #01673921, Dow). When fabricating a master mold, the photoresist is spin-coated on a silicon wafer using a spinner (Cat #KW-4A, SETCAS Electronics) by ramping to the rotational speed of 500 revolutions per minute (rpm) for 5-10s with an acceleration of 100 rpm/s, and then ramping to 2500 rpm at 300 rpm/s acceleration for 30 s, thereby forming a 30 μm-thick photoresist layer. The master mold is surface-modified by silanization using Trichloro (1H, 1H, 2H, 2H-perfluorooctyl) silane (Cat #448931-10G, Sigma-Aldrich) before soft-lithography. During soft-lithography, PDMS is poured onto the silanized silicon wafer, followed by stringent degassing using a vacuum pump (Cat #167300-22, Rocker). The inlet and outlet of the microfluidics are punched on the PDMS microchannel layer prior to bonding to a glass cover slide. Other proper methods to make microfluidic chip are also possible.

FIG. 2B shows velocity distribution in a microfluidic chip 201 derived from Laminar flow velocity simulation under 50 μL/min flow rate (i.e., the flow rate of the fluid sample at the inlet is 50 μL/min) using a commercial software COMSOL. Configuration of microfluidic chip 201 is substantially the same as the microfluidic chip 200 with reference to FIG. 2A. FIG. 2C shows configuration of the microconstrictions that are divided into four groups 232a, 234a, 236a, and 238a, each including nine microconstrictions. Configuration of the thirty-six microconstrictions is same, each having a width of 10 μm, a height of 30 μm, and a length of 60 μm. As can be seen, the velocity across the thirty-six microconstrictions is almost the same, indicating that there are no systematic differences in fluidic features among these microconstrictions. FIG. 2D shows velocity distribution along a direction from the inlet toward the outlet in a certain region including a single microconstriction region 239. As can be seen, cells have larger velocity at the microconstriction region 239 than that before or after the region 239.

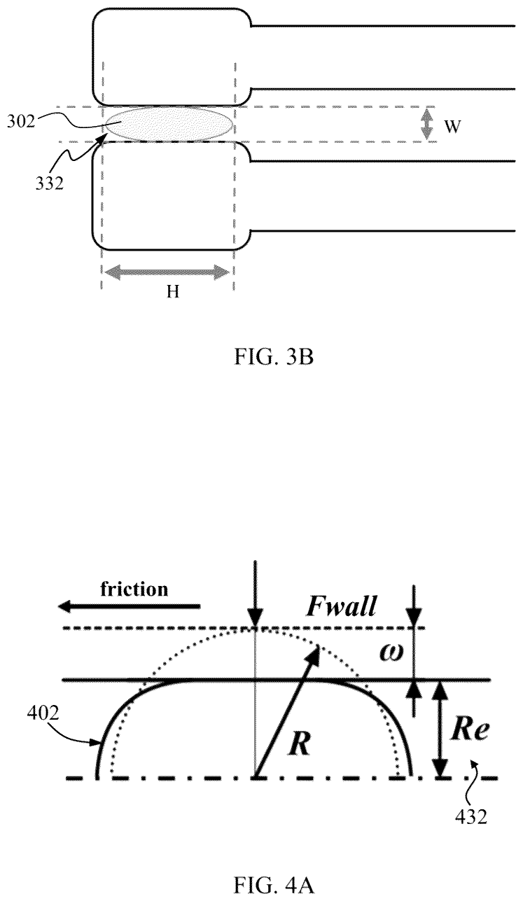

FIG. 3A illustrates a process of a cell 302 travelling through a microconstriction 332 in accordance with certain embodiments. FIG. 3B illustrates the cell 302 of FIG. 3A is fully within the microconstriction 332 and deformed therein.

As illustrated in FIG. 3A, at time T1, the cell 302 is in the main channel or sub-channel but has not reached the microconstriction 332. At time T2, the cell 302 touches the microconstriction 332 and begins to enter the microconstriction 332. T2 is entry time or entering time. At time T3, the cell 302 has fully entered the microconstriction 332 and is deformed. At time T4, the cell 302 has passed and left the microconstriction 332. T4 is leaving time. The time difference (T4−T2) is defined as passage time or transition time. That is, the passage time is the time needed for a cell to pass through a microconstriction, which describes how fast the cell is moving.

As illustrated in FIG. 3B, the limited space of microconstriction 332 elongates the cell 302 in the moving direction to a dimension of H, but squeezes the cell 302 in a direction perpendicular to the moving direction to a dimension of W. The deformation index is a function of H and W, and can be represented as f(H, W). In some embodiments, f(H, W)=(H−W)/(H+W). In some embodiments, the deformation index may be defined in other manner by properly constructing a formula combing of W and H.

Passage time is a motional parameter related to cell's frictions and elastic modulus and is negatively correlated with deformability, while deformation index is a morphological parameter and is positively correlated with deformability. They are good indicators of cell's property, such as invasiveness. For example, cells, such as cancer cells, with higher invasiveness have lower passage time and higher deformation index.

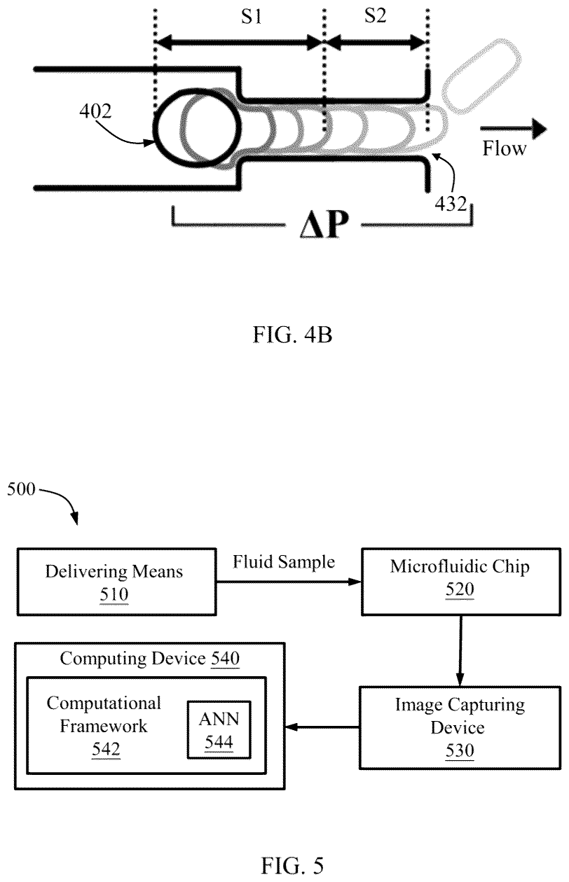

FIG. 4A illustrates a cell 402 deformed in a microconstriction 432 in accordance with certain embodiments. FIG. 4B illustrates a process of the cell 402 of FIG. 4A passing through the microconstriction 432.

As shown in FIG. 4A, when the cell 402 is in the microconstriction 432, the cell 402 is squeezed and deformed by the wall of the microconstriction 432. The pressure or force of the wall is F_{wall}, and when the cell 402 enters, the friction of the wall is friction=μ·F_{wall}, with μ being the friction coefficient of the microconstriction 432. The cell may be considered as incompressible during the relatively short entry time, and thus a parameter εₑ is applied to quantify the maximum deformation rate of cell following Equation (1):

$${ε}_{e} = \frac{R - {R}_{e}}{R} \qquad ( 1 )$$

where R is the radius of the cell, and Rₑ is half of the width of the microconstriction.

After the cell enters the microconstriction, as shown in the FIG. 4B, the pressure on the left side (closer to the inlet) is greater than that on the right side (closer to the outlet), which generates a ΔP, resulting a horizontal force from left to right. After cells entering the microconstriction, the total stage can be divided into a creep stage (S1) and a transit stage (S2).

The creep stage, as a first stage, describes the process from the cell being detected at the entrance of the microconstriction to the cell being entered the microconstriction fully. The time period for the cell during this stage is called creep time, which is represented by t_{creep}. The second stage is the transit stage, which lasts from the cell being fully entered the microconstriction to completely leaving the microconstriction. The time period for the cell during this stage is called transit time, which is represented by tₜᵣₐₙₛᵢₜ. In some embodiments, by collecting data of 526 (i.e., the number of MCF-7 cells is 526) MCF-7 cells and 562 (i.e., the number of MDA-MB-231 cells is 562) MDA-MB-231 cells, the average diameters of MCF-7 and MDA-MB-231 are found to be 28.57 μm and 29.45 μm, respectively. Thus, in some embodiments, the region of the creep stage may be set as 29 μm before and after the entrance of a microconstriction.

By way of example, the cell may be taken as a viscoelastic homogeneous body. Therefore, power-law rheology can be applied to describe changes in cell strain ε over the creep stage (t_{creep}), where t_{creep} is the time of a cell staying in the creep stage, and is called creep time or creeping time. The formula describing the relationship between ε and t_{creep} is shown in Equation (2) as below:

$$ε = \frac{Δ \overline{P}}{{E}_{cell}} {( \frac{{t}_{creep}}{{t}_{0}} )}^{β} \qquad ( 2 )$$

Where ΔP̅=1/t_{creep}∫Δp(t) dt represents the mean pressure difference in the microconstriction, E_{cell} is the Young's modulus of cell, β is the power-law exponent and the value of β, by way of example, can be 0.1˜0.5 for the cell. Since t₀ is the timescale, which can be arbitrarily set to 1 s. As such, Equation (2) can be transformed to Equation (3) as below:

$${t}_{creep} = {( \frac{{ε}_{e} {E}_{c e l l}}{Δ \overline{P}} )}^{\frac{1}{β}} \qquad ( 3 )$$

The cell area or cell's area or cell's size or cell size can be calculated as A_{cell}=π·R². The deformation rate of cell can be rewritten as:

$${ε}_{e} = \frac{R - {R}_{e}}{R} = 1 - \frac{{R}_{e}}{R} = 1 - \frac{{R}_{e} \cdot \sqrt{π}}{\sqrt{{A}_{cell}}} \qquad ( 4 )$$

Set constant

$${c}_{1} = {E}_{cell}^{\frac{1}{β}} / Δ {\overline{P}}^{\frac{1}{β}} ,$$

and c₂=Rₑ·\sqrt{π}. ΔP̅ is the mean pressure drop across the microconstriction, which can be assumed as a constant in each experimental run under the same infusing flow rate (i.e., flow rate of fluid sample at the inlet). Therefore, c₁ can be considered as a measurement that is proportional to the elasticity of the cell, while c₂ is a constant proportional to the width of the microconstriction, where the width is measured in a direction substantially perpendicular to the moving direction of a cell in the microconstriction. Set y=log(t_{creep}). The equation for cell area and creep time can be written as:

$$y = f ( {A}_{cell} ) = {t}_{creep} = {c}_{1} \cdot {( 1 - \frac{{c}_{2}}{{A}_{cell}^{\frac{1}{2}}} )}^{\frac{1}{β}} \qquad ( 5 )$$

The power-law exponent β equal 0, when the material is purely elastic; and the β equal 1, when the material is a purely viscous fluid. For cells, β usually falls within 0.1˜0.5. In some embodiments, the program sets β=0.5 for calculation.

FIG. 5 illustrates a system 500 for determining cell deformability in accordance with certain embodiments.

As illustrated, the system 500 includes delivering means 510, a microfluidic chip 520, an image capturing device 530, and a computing device 540.

The microfluidic chip 520 can be a microfluidic chip as described above with reference to one or more figures. For example, the microfluidic chip 520 includes an inlet, an outlet, a plurality of main channels provided with parallelized microconstrictions for deforming cells, and one or more bypass channels that are independent from the main channels and adjust pressure drop between the inlet and the outlet.

The delivering means 510 delivers, such as infuses or injects or in other proper manners, the cells to the microfluidic chip 520 via the inlet such that the cells are deformed by the parallelized microconstrictions. The flow rate at the inlet can be in a range from 30 μL/min to 80 μL/min, which corresponds to a flow velocity from 0.054 m/s to 0.143 m/s. The flow rate can be other values according to practice needs, such as specific configuration of the system, the property of fluid samples.

In some embodiments, the delivering means 510 includes a syringe pump. The syringe pump pumps a fluid sample including the cells into the inlet of the microfluidic chip 520 such that the cells flow through the main channels under pressure difference between the inlet and the outlet and are deformed when passing through the parallelized microconstrictions. Other proper delivering means are also possible, as long as by means of which, the cells can be conveyed and enter the microfluidic chip 520 for further processing.

The image capturing device 530 collects data of the cells when the cells pass through the parallelized microconstrictions and generate collected data. The collected data can be related to morphological and motional parameters of the cells. The collected data, for example, can be based to calculate passage time and deformation index. The collected data, for example, can be processed for cell detection, tracking, segmentation, and/or classification. In some embodiment, the image capturing device 530 is a camera, such as a high-speed video camera. The image capturing device 530 may make use of other imaging technologies, such as quantitative phase imaging, to fetch high dimensional data.

The computing device 540 communicates with the image capturing device 530 such that the collected data can be received or fetched from the image capturing device 530. The computing device 540 includes a computational framework 542 that is implemented with an ANN 544 that performs various functions, such as determining the cell deformability based on the collected data.

In some embodiments, the ANN 544 includes a cell detector for detecting positions of the cells. By way of example, the cell detector can be selected from a group consisting of YOLOv5, YOLOv6, and YOLOv7.

In some embodiments, the ANN 544 includes a cell tracker for tracking trajectory of the cells. By way of example, the cell tracker can be selected from a group consisting of Deep SORT and Strong SORT. The cell detector and the cell tracker determine passage time for each of the cells that have passed through the microconstrictions.

In some embodiments, the ANN 544 includes a segmentation model for determining deformation index and size for each cell that has passed through the microconstrictions, thereby classifying the cells. The segmentation model, for example, can be ResUnet++ mode or algorithm.

In some embodiments, the computational framework automates generation of a training set by using background subtraction method for training the ANN 544. This is particularly useful in scenarios where a large number of cells may be involved, such that manual generation of training set is laborious, inefficient, time-consuming, and sometimes even impossible.

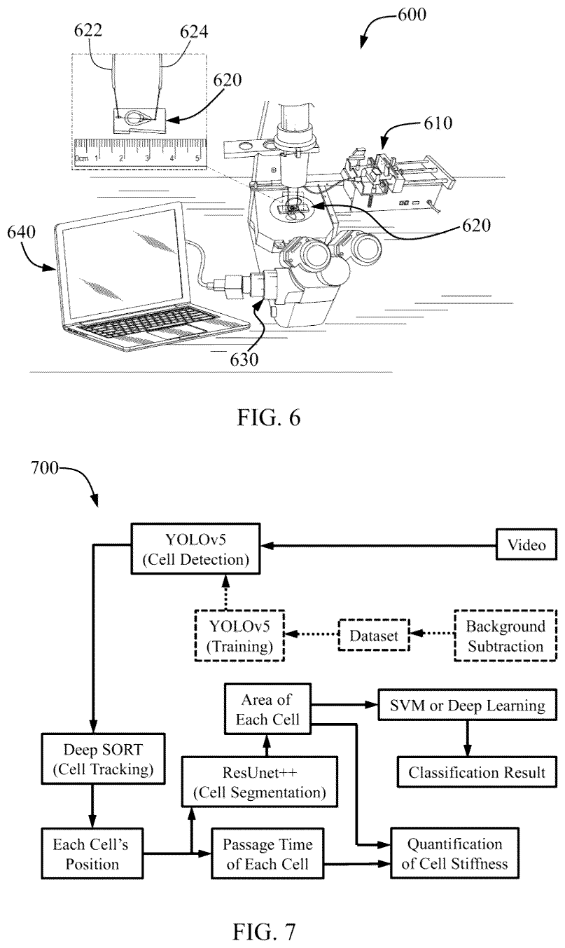

FIG. 6 illustrates a system 600 for determining cell deformability in accordance with certain embodiments. The system 600, for example, can be a specific implementation of the system 500 as described above.

As shown, the system 600 includes a syringe pump 610, a microfluidic chip 620, a camera 630, and a computing device 640. The microfluidic chip 620 is enlarged to show more clearly about its configuration. Two delivering probes or channels 622 and 624 connect to inlet and outlet of the microfluidic chip 620 respectively such that a fluid sample with cells can enter and leave the microfluidic chip 620.

FIG. 7 shows a diagram 700 illustrating functioning of the computational framework in accordance with certain embodiments. The functioning or operation, for example, can be performed by the system 500 or 600 as described above.

In the present embodiments as shown, YOLOv5 and Deep SORT are used. This is for illustrating purpose only. In some embodiments, YOLOv6 or YOLOv7 is used to replace YOLOv5. In some embodiments, Strong SORT is used to replace Deep SORT.

As illustrated and by way of example, one or more training sets or datasets can be automatically generated, using background subtraction method and without human intervention, for training YOLOv5 model.

The background subtraction method can be implemented based on OpenCV2. The algorithm uses each background pixel modelled by a mixture of K Gaussian distributions (K=3 to 5). The probability of a certain pixel can be written as:

$$p ( {x}_{N} ) = {\sum }_{j = 1}^{K} {w}_{j} η ( {x}_{N} ; {θ}_{j} ) \qquad ( 6 )$$

Where p is a probability distribution, x_{N} is the value of a certain pixel at time N, wⱼ is a weight parameter of the K Gaussian component, and η(x_{N}; θⱼ) is the Normal distribution of K Gaussian component. The first B distribution is used as a model of the background of the scene, and the equation can be written as:

$$B = arg min ( {\sum }_{j = 1}^{b} {w}_{j} > T ) \qquad ( 7 )$$

Where T represents threshold and is the minimum fraction of the background model. It may be the minimum prior probability that the background is in the scene. The background subtraction consists of two parts: background initialization and background update.

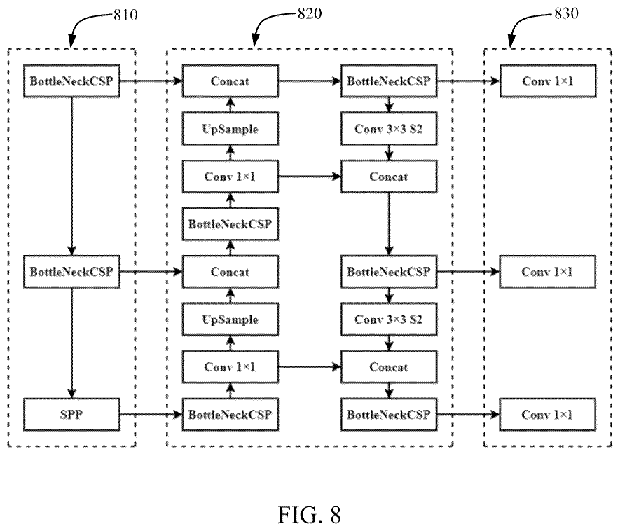

YOLOv5 is a real-time object detection algorithm that divides images into a grid system. It consists of three parts: backbone 810, neck: PANet 820 and head 830, as shown in FIG. 8, where CSP represents cross stage partial network, Conv represents convolutional layer, SPP represents spatial pyramid pooling, and Concat represents concatenate function. The images are input to CSPDarknet for feature extraction, and then the obtained data are fed to PANet for feature fusion. Finally, the algorithm outputs prediction from the head part. Optionally, the algorithm uses mosaic data augmentation to improve the number of training data.

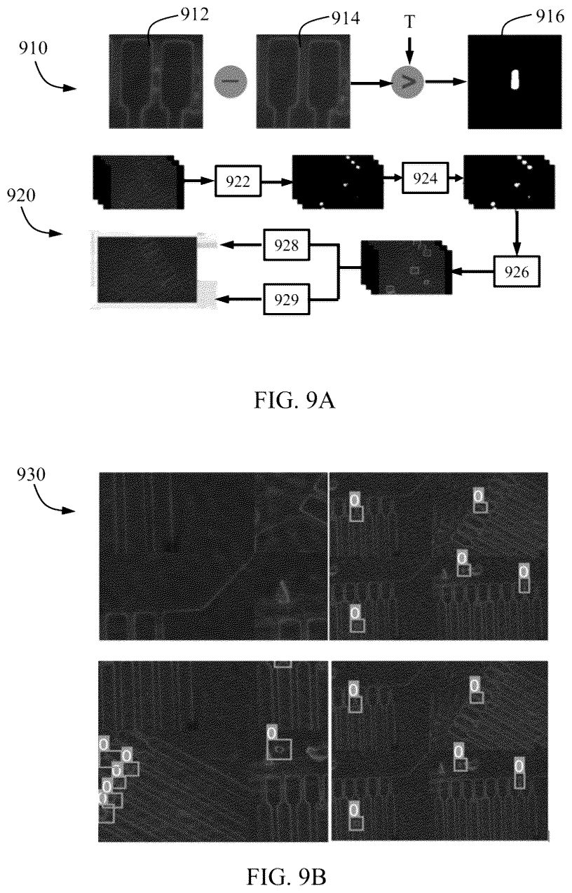

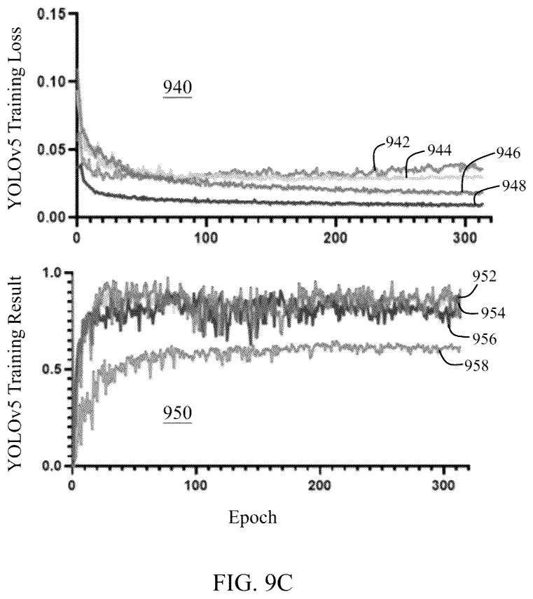

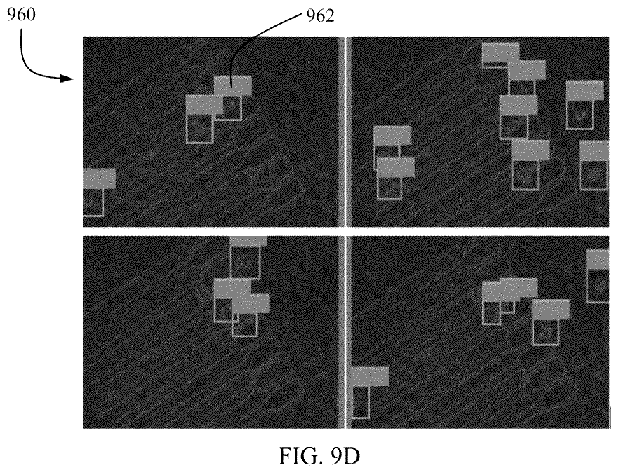

Training of YOLOv5 model is illustrated in FIGS. 9A-9D. FIG. 9A illustrates a graphical principle of background subtraction method (upper panel as indicated by 910) and an overview of training dataset creation for YOLOv5 (lower panel as indicated by 920). FIG. 9B illustrates images 930 in the training set after being processed for mosaic augmentation. FIG. 9C illustrates training loss, precision, recall and mean average precision (mAP) of YOLOv5. FIG. 9D illustrates detection of cells by established or trained YOLOv5 model.

As illustrated, the background subtraction method calculates the foreground mask 916 by performing a subtraction between the current frame 912 and a background model 914, containing the static part of the scene or, more in general, that can be considered as background given the characteristics of the observed scene. The subtracted value (i.e., the current frame minus the background model) is compared with a threshold (T). For example, in the foreground mask 916, when the subtracted value is greater than the threshold, the cells or other moving areas in the image will be displayed in white, and other static or non-moving areas will be displayed in black. The threshold is a preset parameter in Open Source Computer Vision Library (OpenCV), which is based on createBackgroundSubtractorMOG2 function. The threshold can be adjusted according to the captured video or images. For example, in some embodiments, the threshold can be set as 16.

For training of YOLOv5 model, initial images are processed by the background subtraction method 922, and then optionally, undergo dilation 924. Dilation is a function in OpenCV. The bright regions of the cells dilate around the black regions of the background. After dilation, the foreground mask gets a smoother white mark which is easier to process. The images after dilation can be used to obtain annotations at block 926. For example, findContours and boundingRect in OpenCV can be used to obtain the annotations. The program can automatically read x, y, w, h (i.e., position x, position y, cell's width and cell's height) of all white areas in foreground mask. The annotations 928 and images 929 as finally obtained are used to train YOLOv5. In some embodiments, the annotations 928 and images 929 are obtained by employing open-source programs, such as Labellmg.

FIG. 9B shows images 930 in a training set to which mosaic augmentation is applied to improve robustness of YOLOv5 model. Mosaic augmentation combines four training images into one in certain ratios (instead of only two in CutMix). FIG. 9C shows training loss, precision, recall, and mean average precision (mAP) of YOLOv5 model. Graph 940 shows train/box_loss 946, train/obj_loss 948, val/box_loss 944, and val/obj_loss 942, while graph 950 shows metrics/precision 952, metrics/recall 956, metrics/mAP_0.5 954, and metrics/mAP_0.5:0.95 958.

The term “train/box_loss” refers to loss on the training set for bounding box predictions. The term “train/obj_loss” refers to loss on the training set for objectness predictions. The term “val/box_loss” refers to loss on the validation set for bounding box predictions. The term “val/obj_loss” refers to loss on the validation set for objectness predictions. That is, the terms “train/box_loss” and “val/box_loss” refer to the “bounding box loss” on the training and validation sets, respectively. This loss is a measure of how well the model is able to predict the location of objects in the image, as represented by their bounding boxes. The terms “train/obj_loss” and “val/obj_loss” refer to the “objectness loss” on the training and validation sets, respectively. This loss is a measure of how well the model is able to predict whether an object is present in a given bounding box. The term “epoch” refers to single pass through the entire training dataset during training. For example, during training, the YOLOv5 model processes each training example in the dataset and updates its parameters based on the error it makes in predicting the object locations and class probabilities. The number of epochs specifies the number of times the model will iterate through the entire training dataset during the training process.

The term “metrics/precision” refers to proportion of true positive predictions made by the model, out of all positive predictions made by the model. The term “metrics/recall” refers to proportion of true positive predictions made by the model, out of all actual positive instances in the dataset. The term “metrics/mAP_0.5” refers to Mean Average Precision at IoU threshold 0.5. It is a measure of the model's overall performance on the object detection task. It takes into account both the precision and recall of the model, as well as the overlap between the predicted bounding boxes and the ground truth bounding boxes (as measured by the IoU metric). The term “metrics/mAP_0.5:0.95” refers to Mean Average Precision at IoU thresholds 0.5 and 0.95. This is same as metrics/mAP_0.5, except with an additional IoU threshold of 0.95. The IoU threshold determines the level of overlap required between the predicted bounding box and the ground truth bounding box in order for the prediction to be considered as a true positive. A higher IoU threshold results in a stricter evaluation, while a lower IoU threshold results in a more lenient evaluation.

FIG. 9D shows images 960 illustrating the result of detection of cells by the established YOLOv5 model that has been trained. Cell 962 is a representative cell that is detected. As can be seen, the constructed or trained YOLOv5 model presents good performance in cell detection.

With reference to FIG. 7 again, by way of example, Deep SORT is used as a cell tracker for tracking cells. This is for illustrating purpose only. In some other embodiments, other algorithms can be used, such as Strong SORT.

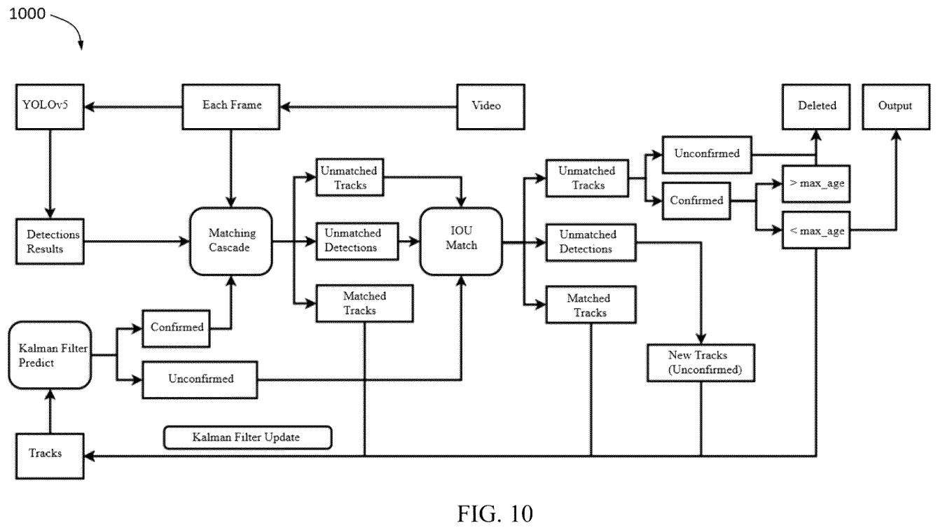

Deep SORT is an algorithm that can be used in multi-object tracking and is a detection-based tracking method. The overview model architecture is illustrated in diagram 1000 of FIG. 10. The overall processes in Deep SORT includes the follows: 1) Kalman filter predicting cell tracks; 2) using the Hungarian algorithm to match the predicted tracks with the detections in the current frame (matching cascade and IOU assignment); 3) Kalman filter update; and 4) calculating the crossing time for each cell.

Kalman filtering is one of the algorithms in the Deep SORT target tracking algorithm. Kalman filtering is mainly to accurately predict the tracking target position based on the movement state of the object, which, based on input data, makes it possible to estimate the total system state.

Deep SORT uses a linear quadratic estimation to estimate the position of each object from the current frame to the next frame. The algorithm uses an eight-dimensional space vector to describe the movement state of an object, which shows in the equation below:

$$x = {[ u , v , r , h , {u}^{′} , {v}^{′} , {r}^{′} {h}^{′} ]}^{T} \qquad ( 8 )$$

Where u, v are the horizontal and vertical coordinates of the bounding box. r, h are the aspect ratio and the height of the target bounding box, and u′, v′, r′, h′ are derivatives of u, v, r, h, which means the velocity of each variable.

The value of x at time k of is estimated from the state at k−1 according to:

$${x}_{k \vert k - 1} = {F}_{k} {x}_{k - 1 \vert k - 1} + {B}_{k} {u}_{k} \qquad ( 9 )$$

Where x_{k|k-1} means a posteriori state estimate at time k and included at time k−1. Fₖ is the state transition model, and Bₖ is the control-input model which is applied to the control vector uₖ at time k.

Predicted estimate covariance is written as follow:

$${P}_{k \vert k - 1} = {F}_{k} {P}_{k - 1 \vert k - 1} {F}_{k}^{T} + {Q}_{k} \qquad ( 10 )$$

P_{k|k-1} is a posteriori estimate covariance matrix, and Qₖ is the covariance of the process noise.

The updated equation of the algorithm area as follows:

$${K}_{k} = {P}_{k \vert k + 1} {H}_{k}^{T} \cdot {( {H}_{k} {P}_{k \vert k - 1} {H}_{k}^{T} + {R}_{k} )}^{- 1} \qquad ( 11 )$$

$${\hat{x}}_{k \vert k - 1} = {\hat{x}}_{k \vert k - 1} + {K}_{k} \cdot ( {z}_{k} - {H}_{k} {\hat{x}}_{k \vert k - 1} ) \qquad ( 12 )$$

$${P}_{k \vert k} = ( I - {K}_{k} {H}_{k} ) {P}_{k \vert k - 1} \qquad ( 13 )$$

Where Hₖ is the observation model, and Rₖ is the noise-dependent covariance.

The Deep SORT algorithm uses the Hungarian algorithm to match the prediction box from the Kalman filter and the detection target from the YOLOv5 algorithm. The Hungarian method is a combinatorial algorithm that can predict primal-dual methods.

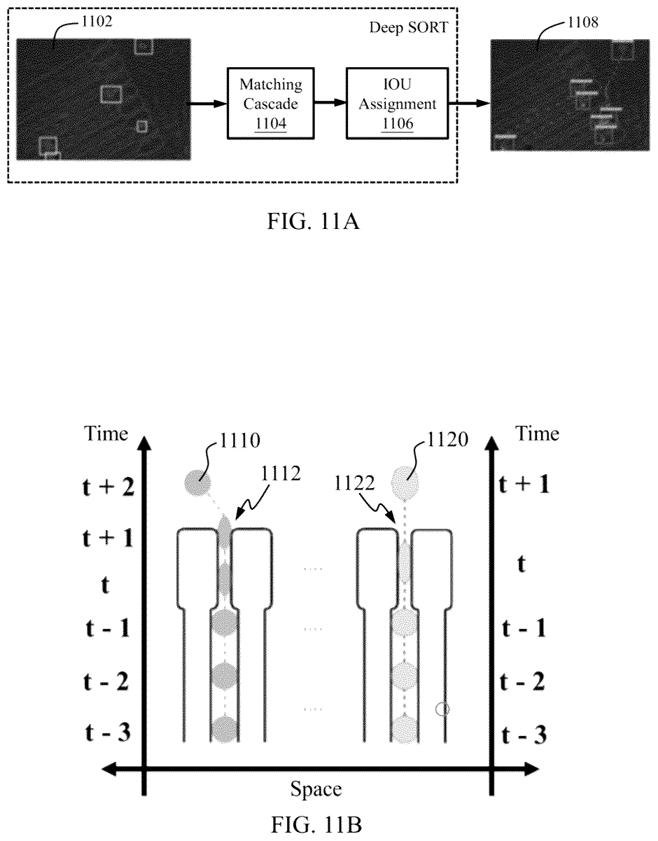

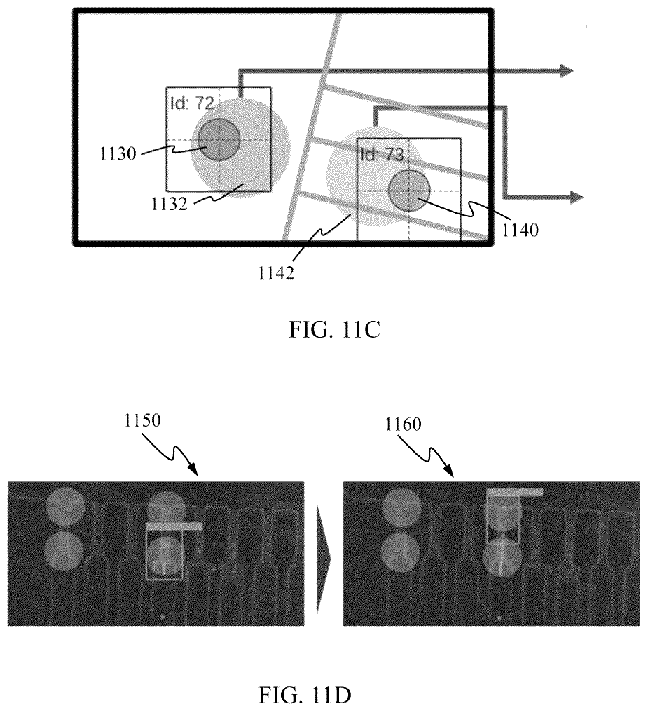

Regarding cell tracking by Deep SORT in FIG. 7, FIGS. 11A-11D present illustrations in this regard. FIG. 11A shows a brief demonstration of cell tracking based on Deep SORT. Cell tracking in the conjugation of YOLOv5-based object detection are applied to assign IDs to cells and quantify their passage time. Deep SORT achieves the tracking based on intersection over union (IOU). Images and annotations 1102 go through matching cascade 1104 and IOU assignment 1106, and result in resulted image 1108 with cells tracked. The rectangles in image 1108 indicate the tracked cells.

FIG. 11B illustrates two types of cells with different deformability are differentiated by passage time. The two types of cells in the present embodiments are MCF-7 1110 and MDA-MB-231 1120, as an example for illustrating purpose only. MCF-7 1110 passes through a channel 1112, while MDA-MB-231 1120 passes through a channel 1122. As can be seen, MDA-MB-231 1120 moves faster and has shorter passage time.

FIG. 11C illustrates application of focusing areas for passage time quantification. Calculation of passage time needs to determine the time point of a cell when entering and leaving a microconstriction. In this regard, focusing areas are provided. A focusing area can be disposed at an entrance of the microconstriction. Once the cell is detected in the focusing area, the time point (Tₑₙₜₑᵣ) and identity of the cell are recorded. A focusing area can be disposed at an exit of the microconstriction. Once the cell is detected in the focusing area, the time point (Tₗₑₐᵥₑ) and identity of the cell are recorded. Assuming Tₑₙₜₑᵣ and Tₗₑₐᵥₑ for a same cell (i.e., discovered as having a same identity) passing through the microconstriction are obtained, the passage time will be calculated as Tₗₑₐᵥₑ−Tₑₙₜₑᵣ. FIG. 11C illustrates two cells 1130 and 1140, with an ID number 72 and 73 respectively, are detected in focusing areas 1132 and 1140 respectively.

FIG. 11D shows actual images illustrating how the analyzing program runs. Image 1150 shows how the cell is captured and identified when it enters the microconstriction. Image 1160 shows how the cell is captured and identified when it leaves the microconstriction. The rectangular boxes surrounding the cells display location and identity of the tracked cells.

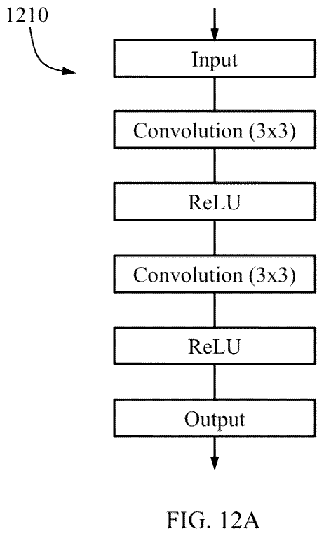

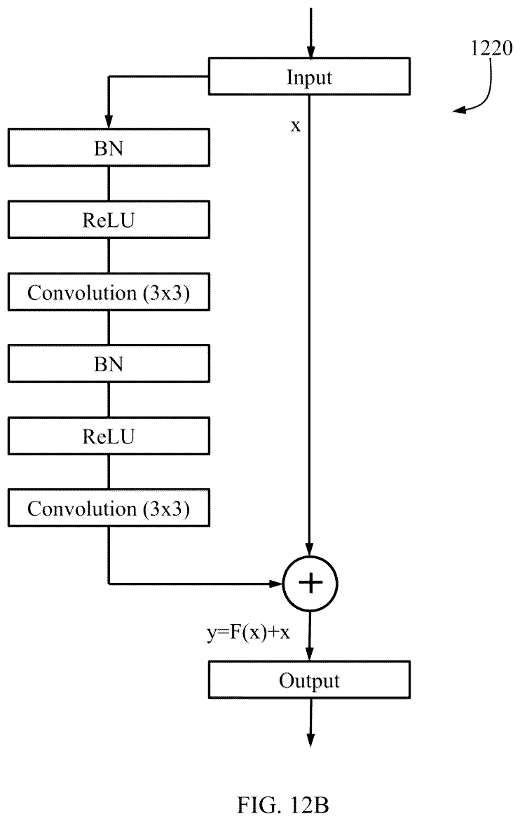

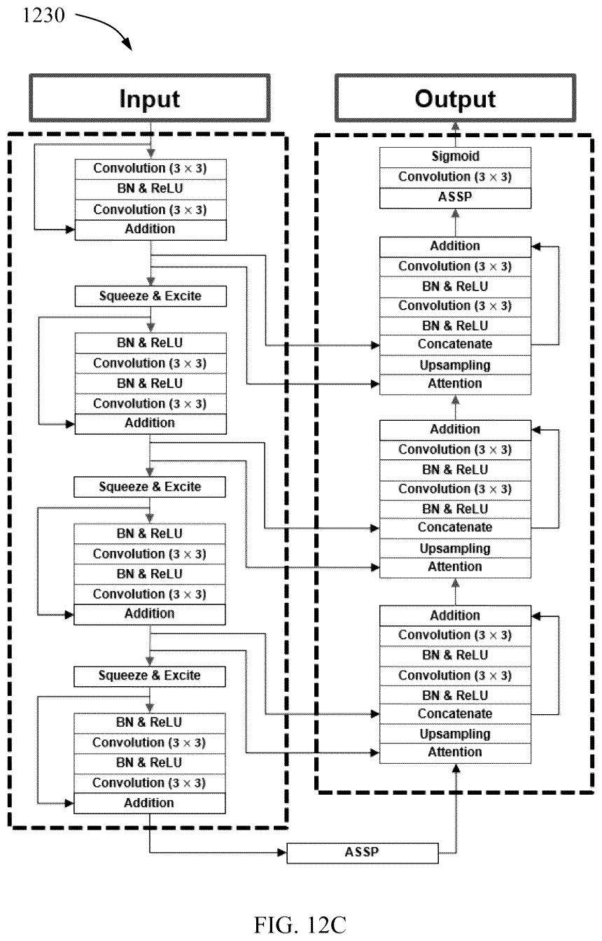

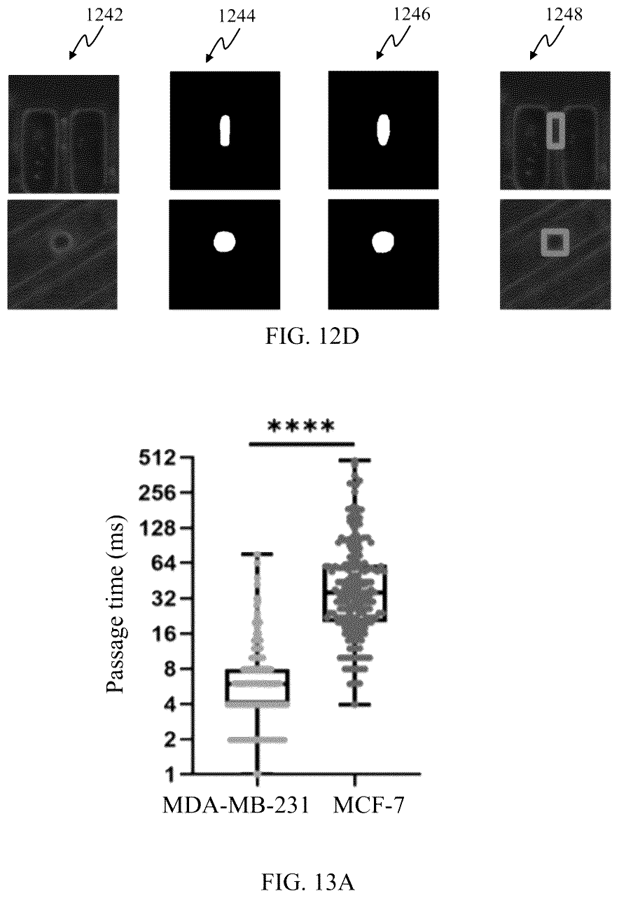

With reference to FIG. 7 again, ResUnet++ is used for cell segmentation. By way of example, cells' minimal enclosing rectangles and areas (A_{cell}) can be obtained. FIGS. 12A-12D present illustrations in this regard.

FIG. 12A is a diagram 1210 showing blocks unit used in Unet. FIG. 12B is a diagram 1220 showing residual unit with identity mapping used in ResUnet. In one or more embodiments as described herein, ResUnet++applies residual unit with identity mapping. FIG. 12C is a diagram 1230 showing an architecture of ResUnet++. FIG. 12D shows segmentation of a cell from its original picture based on the trained ResUnet++ model with the architecture of FIG. 12C. The images indicated by 1242, 1244, 1246, and 1248 refer to original picture or image, prediction result, ground truth, and cell marking by using minimal enclosing rectangles respectively. FIGS. 12A-12D demonstrates good segmentation results by using the trained ResUnet++ model.

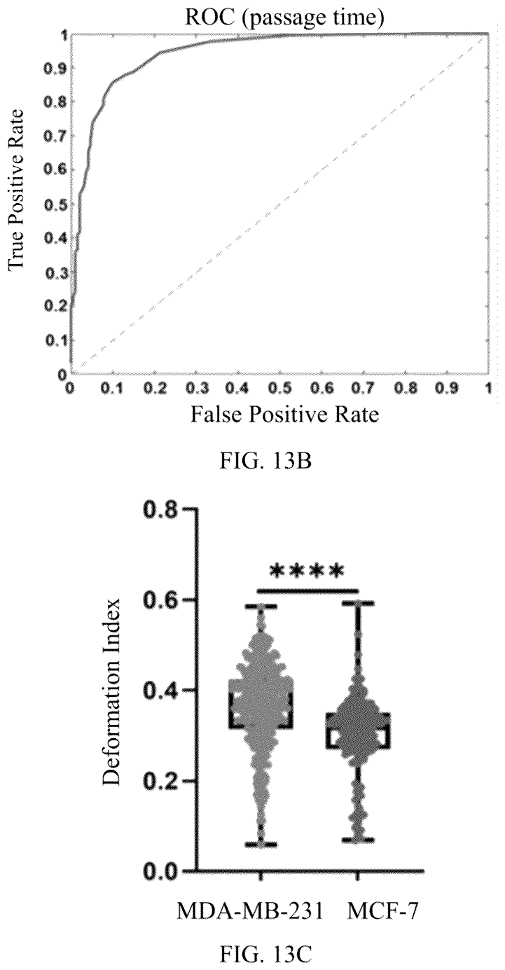

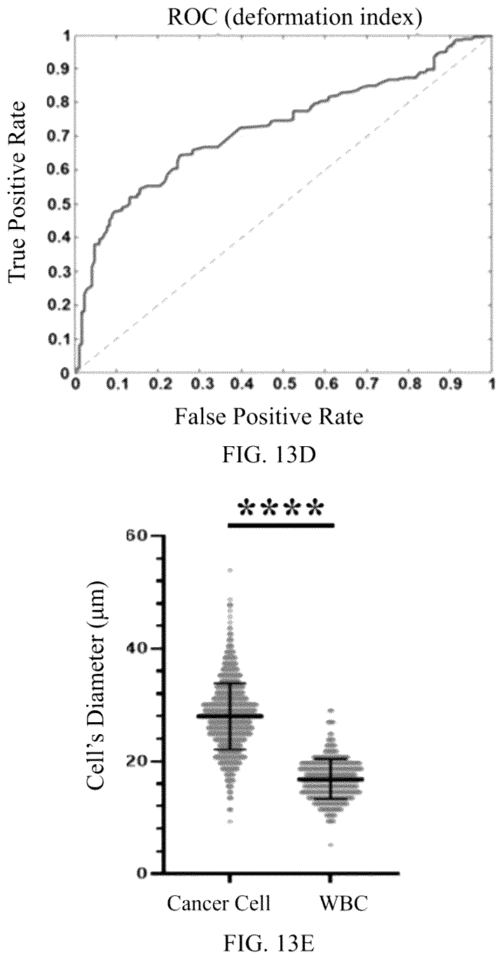

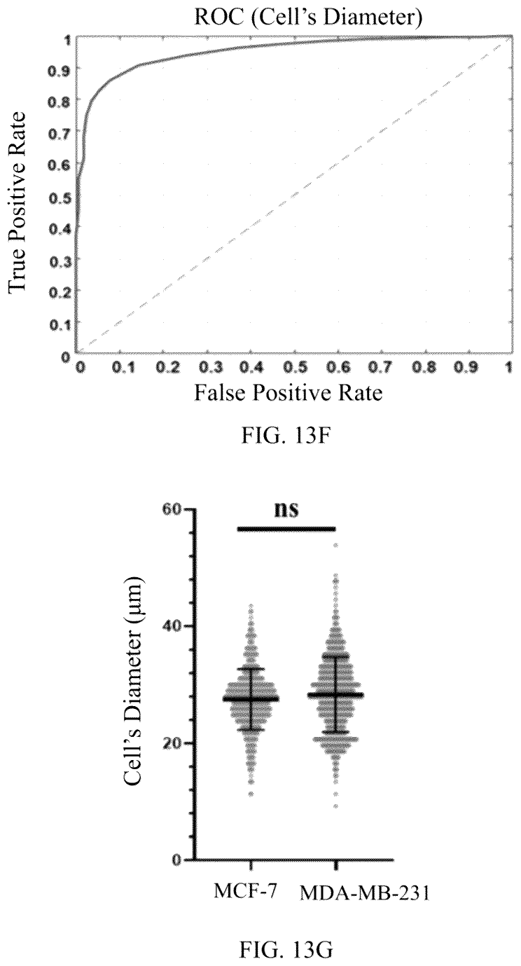

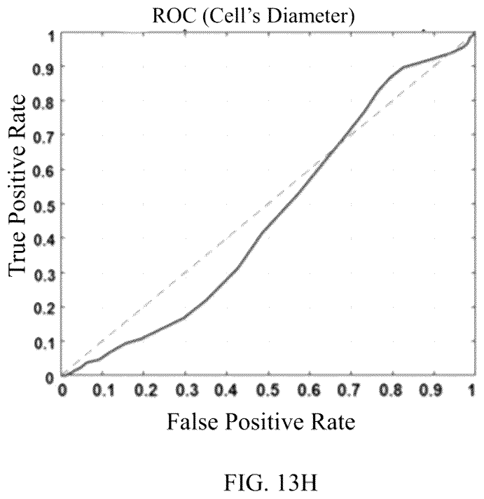

FIGS. 13A-13H shows classification of blood cells and cancer cells varied in metastatic potentials.

FIGS. 13A-13D relates to the two types of cells used for illustrating purpose only, which are MCF-7 and MDA-MB-231 (ATCC). MCF-7 originates from estrogen and progesterone receptor positive breast cancer subtypes, while MDA-MD-231 originates from a more aggressive triple-negative subtype. Both cell lines are adhesion cells cultured by high-glucose Dulbecco's Modified Eagle Medium (DMEM) (Cat #10566-016, Thermo Fisher) with 10% fetal bovine serum (FBS) (Cat #10270106, Thermo Fisher) in a 37° C. incubator (5% CO2). Before processing with microfluidic chips, cells are detached by trypsin (Cat #R001100, Thermo Fisher), diluted to 3˜5×10⁵ cells/mL in DMEM culture media with 0.1% (v/v) Pluronic F-127 surfactant (Cat #24040-032, Thermo Fisher). Before each run, the microfluidic chip is washed with 2.5% bovine serum albumin under a flow rate of 20 μL/min for 10 minutes, followed by washing with phosphate-buffered saline under a flow rate of 50 μL/min for 2 minutes. Ensure that no air bubbles are remained in microchannels before priming cells into the chip. After washing, cells are primed into the microfluidics under a 50 μL/min flow rate. A high-speed camera (MV-A5031MU815, Dahua Technology) operated at 988 frame-per-second (fps) is mounted to the microscopy to record images for analysis.

FIG. 13A compares passage time between MCF-7 and MDA-MB-231. Statistical significance is calculated by two-tailed Student's t-test (P-value <0.05: *; <0.01: **; <0.001: ***; <0.0001: ****). FIG. 13B shows passage time receiver operating characteristic (ROC) curve of MCF-7 and MDA-MB-231. FIG. 13C compares deformation index between MCF-7 and MDA-MB-231. FIG. 13D shows the deformation index ROC curve of MCF-7 and MDA-MB-231.

As shown in FIGS. 13A and 13C, the passage time of MCF-7 is significantly longer than that of MDA-MB-231, while the deformation index of MCF-7 is significantly smaller. This indicates that MCF-7 has higher stiffness and shear modulus, as well as longer cell retention and weaker metastatic potential than MDA-MB-231. On the contrary, MDA-MB-231 has significantly lower passage time and higher deformation index than those of MCF-7 (see FIG. 13C), reflecting its higher metastatic potential due to its better ability to deform and squeeze through the vessel wall.

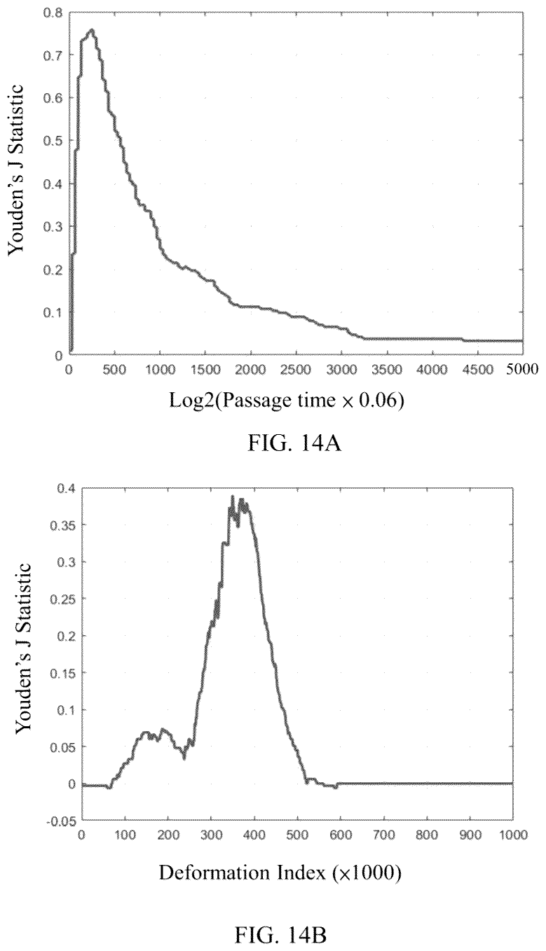

To evaluate the classifying efficiency of the two indexes measured by the system, ROC curves of the two indexes are plotted in classifying MCF-7 and MDA-MB-231 cell lines (FIG. 13B and FIG. 13D). Based on the ROC curves, an optimal threshold for each index in classification is obtained by computing Youden's J statistic (see FIGS. 14A-14B). According to the confusion matrix of the two indexes, classifying the two cell types based on the optimal thresholds of passage time and deformation index achieve an accuracy of 87.8% and 67.7%, respectively (see FIG. 13B and FIG. 13D).

In samples of liquid biopsy, cancer cells are usually mixed with white blood cells. A potential way of distinguishing cancer cells from white blood cells is gating based on cells' diameters. In order to confirm whether the system as described according to one or more embodiments can effectively distinguish cancer cells from white blood cells, pure samples of white blood cells are run on the system, which uses the computational framework to calculate these cells' diameters.

FIG. 13E compares cell diameters between cancer cells and WBCs. FIG. 13F shows cell diameter ROC curve of cancer cells and WBCs. FIG. 13G compares cell diameter between MCF-7 and MDA-MB-231. FIG. 13H shows cell diameter ROC curve of MCF-7 and MDA-MB-231. As shown in FIG. 13E and FIG. 13F, by gating with a threshold in cells' diameters calculated by the computational framework, the system achieves a high accuracy of 89.5% in classifying cancer cells from white blood cells. In the meantime, there is no difference in mean diameter between MCF-7 and MDA-MB-231 (FIGS. 13G and 13H). This result further demonstrates the great potential of the system as described herein according to one or more embodiments in processing samples of liquid biopsy.

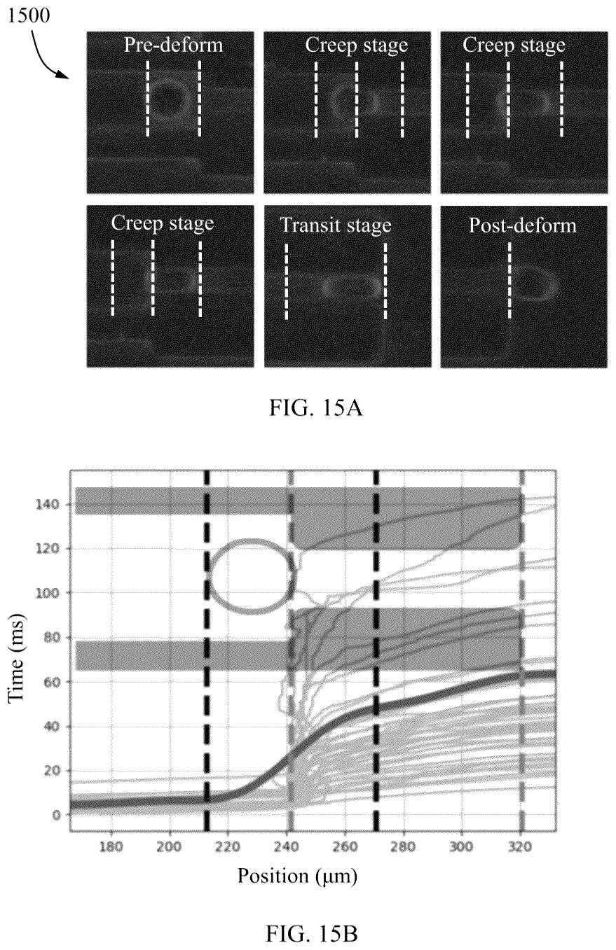

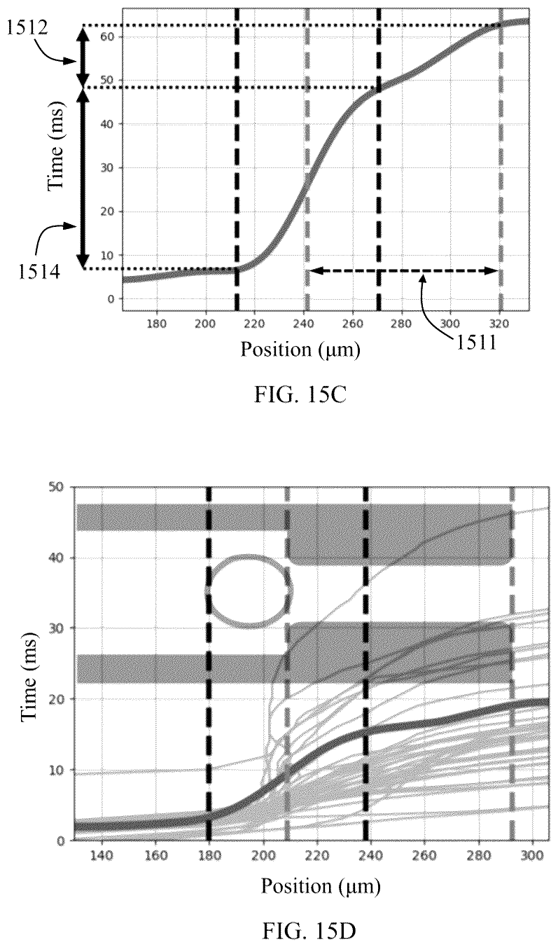

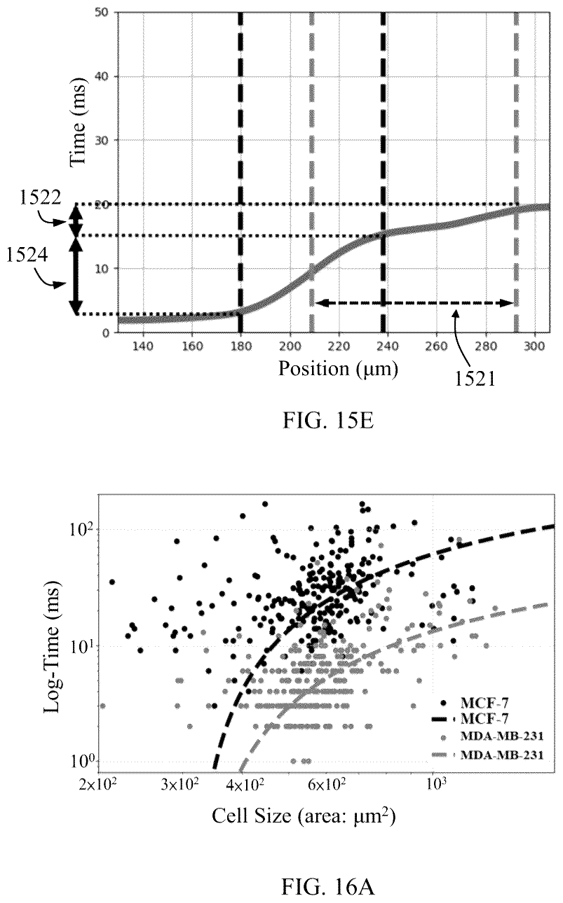

FIG. 15A-15E illustrate qualification of cells' stiffness by trajectory and deformation analysis. Trajectory analysis of position and time of MCF-7 and MDA-MB-231 cells are used for illustrating purpose only. FIG. 15A are images 1500 showing deformation of cells after entering the microconstriction and then passes through the same. FIG. 15B illustrates trajectory of MCF-7 cells in the form of position vs time curve when traveling through the microconstriction. A schematic diagram of microconstriction is shown in the line plot to demonstrate the correspondent relations between the position value on x-axis and the actual position in a microconstriction. The range of y axis is from 0 to 140 microsecond (ms). FIG. 15C shows average time vs position curve of MCF-7 cells through the microconstriction, the dimension of which is indicated by 1511, with creep time as indicated by 1514 and transit time as indicated by 1512 and the range of y axis is from 0 to 65 ms. FIG. 15D illustrates trajectory of MDA-MB-231 cells in the form of position vs time curve when traveling through the microconstriction. The range of y axis is from 0 to 50 ms. FIG. 15E shows average time vs position curves of MDA-MB-231 cells through the microconstriction, the dimension of which is indicted by 15211, with creep time (as indicated by 1524) and transit time (as indicated by 1522) and the range of y axis is from 0 to 50 ms. The gray vertical dotted lines in FIG. 15B-15E represent the entrance and exit of the microconstriction. The distance between the left black vertical dashed line and the adjacent gray vertical dashed line represents the average cell diameter.

By presenting passage time and diameter of the tested cells on two-dimensional plots, the system can effectively distinguish the two cancer cell types into two populations. The passage time is correlated with cell size, and in some embodiments, a constant related to elastic modulus is applied to evaluate the stiffness of the two cell types. Therefore, t_{creep} of the two cell types obtained from their traveling trajectories is applied to calculate their cl indexes. According to the microconstriction width, the specific value of c₂ constant for the microfluidic chip can be obtained: c₂=Rₑ·\sqrt{π}=10 μm·\sqrt{π}=17.7245 μm. Based on c2, c1 of the two cell types and be calculated, which is a direct quantification to represent stiffness of a group of cells, by Equation (5) according to the collected data of t_{creep}. In this way, the c1 indexes for MCF-7 and MDA-MB-231 are calculated, with cl of MCF-7 being much higher than MDA-MB-231 as shown in Table 1 below, indicating the higher stiffness of MCF-7.

TABLE 1
The value of c1 and c2 fitting by MATLAB

| | | |
|---|---|---|
| Cell type | c1 | c2 |

| | | | |
|---|---|---|---|
| MCF-7 | 312.7 | (288.1, 337.3) | 17.7245 μm |
| MDA-MB-231 | 67.85 | (62.37, 73.33) | 17.7245 μm |

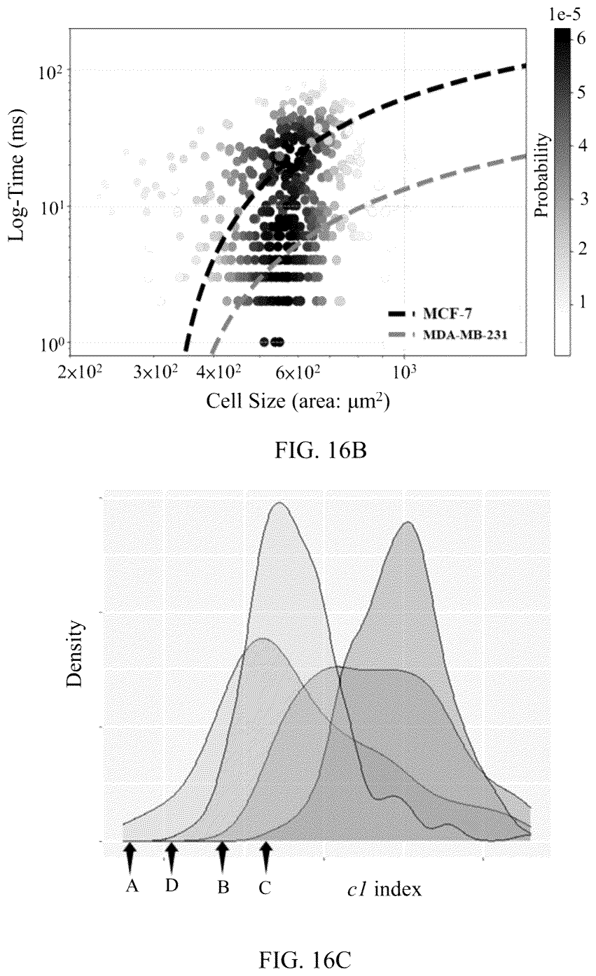

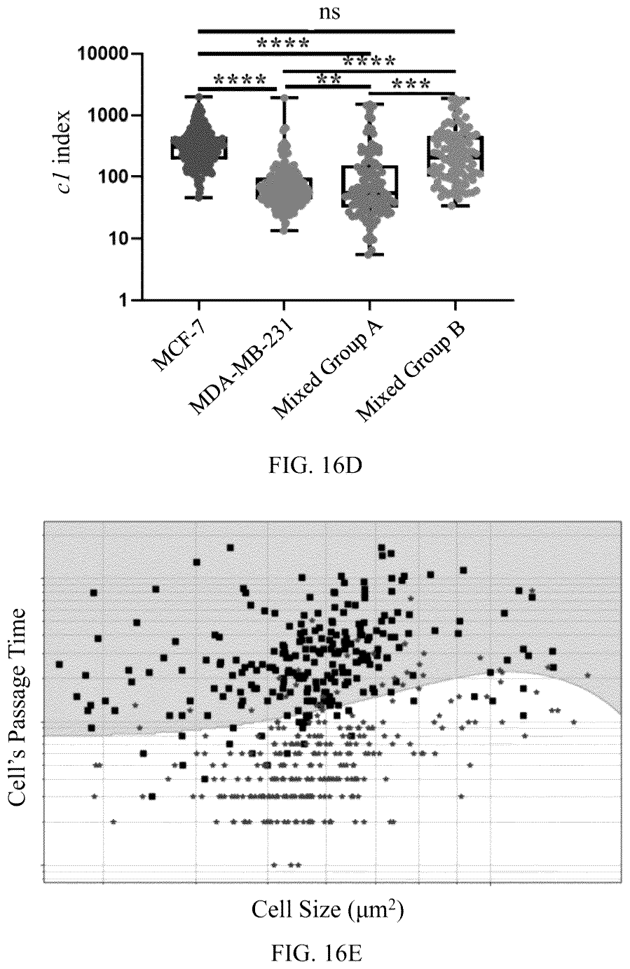

Evaluating metastatic potential of heterogeneous mixed samples is shown in FIGS. 16A-16E.

FIG. 16A illustrates passage time (in log 10 scale) vs cell size (represented by cell's area) for MCF-7 and MDA-MB-231 cells, where data points in black and grey represent MCF-7 and MDA-MB-231 cells respectively, and dash lines in black and grey for MCF-7 and MDA-MB-231 cells respectively are theoretical cell size vs t_{creep} curves calculated by Equation (5) using the calculated cl indexes. FIG. 16B illustrates passage time (in log 10 scale) vs cell size (represented by cell's area) for MCF-7 and MDA-MB-231 cells, where data points in black and grey represent MCF-7 and MDA-MB-231 cells respectively according to their local density, and dash lines in black and grey for MCF-7 and MDA-MB-231 cells respectively are theoretical cell size vs t_{creep} curves calculated by Equation (5) using the calculated cl indexes. FIG. 16C is Kernel density estimation plots showing distribution of cl indexes for MCF-7 (represented by C), MDA-MB-231 (represented by D), and mixed group A (represented by A), and represented by B (represented by B). The ratio for the mixed group A is MCF-7:MDA-MB-231=1:4, while ratio for the mixed group B is MCF-7:MDA-MB-231=4:1. FIG. 16D shows values of cl index for MCF-7, MDA-MB-231, and the mixed group A and the mixed group B, where statistical significance is calculated by two-tailed Student's t-test (P-value >0.05: ns; 0.05: *; <0.01: **; <0.001: *<0.0001: ****). FIG. 16E shows classification result of support vector machine (SVM) of the MCF-7 and MDA-MB-231, where solid squares and stars indicate different types of cells.

In actual clinical settings, tumors are usually heterogeneous tissues, meaning that cancer cells inside the same tumor might be having different phenotypes and metastatic potentials. In order to demonstrate that the system is capable for identifying the phenotypic heterogeneity of cancer cells in terms of metastatic potential when handling actual clinical samples, experiments are conducted with two groups of mixing samples (group A and group B). After processing these samples with our system, the c1 index is calculated for each detected cell according to Equation (5). According to the kernel density estimation (KDE) plot (FIG. 16C), groups A and B have different distribution of c1 index, demonstrating that the two groups have different population of cells. Meanwhile, although the KDE plot of group A and group B have peaks similar to their major cell type components (group A: MDA-MB-231; group B: MCF-7), their distributions are shifted slightly. Group A shows improved proportion of cells with c1 indexes higher than 13.74 (median of cl index of MCF-7) compared with pure MDA-MB-231 (0.16 versus 0.12), while Group B shows improved proportion of cl indexes lower than 8.02 (median of c1 index of MDA-MB-231) compared with pure MCF-7 (0.23 versus 0.05) (FIG. 16C). These shifts in distribution can be further demonstrated by Student's t-tests (FIG. 16D). With the proportion of metastatic cancer cells increased, the c1 index of the cell population is decreased. This is reflected by the greater statistical difference in comparing group MCF-7:MDA-MB-231 (p-value <0.0001) than in comparing group MCF-7:Group A (p-value=0.0071) (FIG. 16D). These results indicate that the changes in metastatic potentials of samples are reflected on the distribution of their c1 indexes, and the systems as described herein according to one or more embodiments are capable of evaluating the metastatic potentials for samples with heterogeneous populations.

An SVM is a supervised machine learning model that uses classification algorithms for two-group classification problems. After giving an SVM model sets of labeled training data for each parameter, the algorithm is able to categorize different cell types. The classification result of SVM based on the passage time of the cell and the area of the cell is 89.260% (FIG. 16E).

A desirable microfluidics-based deformability cytometry is expected to be a sensitive, low-cost, high-throughput, easy-to-operate, and massively producible systems for applications outside research laboratories. However, these criteria are usually mutually exclusive in engineering implementation, and the existing technologies are unfavorable in one aspect or another. For example, although xDC and sDC systems are the most high-throughput models for deformability measurement before the present disclosure, their operation requires the supports of expensive high-speed cameras to be operated under very high frame rates (>2000 fps). In contrary, cDC can be supported with much cheaper cameras under <1000 fps, but the throughput is much lower.

The systems according to one or more embodiments as described herein achieve a balanced and improved performance. For example, Table 2 shows a comparison in performance between the systems according to one or more embodiments (Ref P) and some existing technologies (Ref R1-R7).

TABLE 2
comparison of current systems for deformability cytometry

| | | | | | Reflection | |
|---|---|---|---|---|---|---|
| | | | | | of cells' | |
| | System | Location of | | | migration and | |
| Type | category | measurement | Throughput | Cost | retention | Ref |
| Conventional | Atomic force | At the cell surface | 1~20 cells | High | Not sensitive | R1 |
| method | microscopy | | per hour | | | |
| Conventional | Optical | Whole suspended | 60-300 cells | High | Not sensitive | R2 |
| method | stretching | cell | per hour | | | |
| Conventional | Micro- | Whole suspended | 20 cells | Low | Sensitive | R3 |
| method | aspiration | cell | per hour | | | |
| Microfluidics- | xDC | Whole suspended | >1,000 cells | High | Not sensitive | R4 |
| based method | | cell | per second | | | |
| Microfluidics- | sDC | Whole suspended | >100 cells | Middle | Not sensitive | R5 |
| based method | | cell | per second | | | |
| Microfluidics- | cDC | Whole suspended | >1,000 cells | Low | Sensitive | R6 |
| based method | | cell | per hour | | | |
| Microfluidics- | Parallelized | Whole suspended | 6,000-10,000 | Low | Sensitive | R7 |
| based method | cDC | cell | cells per hour | | | |
| Microfluidics- | Present | Whole suspended | ~25,000 cells | Low | Sensitive | P |
| based method | system | cell | per minute | | | |

R1-R7 refer to the sources of results as set forth below. R1: Liu, X. Y., Wei, Y. H., Li, W., Li, B. & Liu, L. Cytoskeleton induced the changes of microvilli and mechanical properties in living cells by atomic force microscopy. J Cell Physiol 236, 3725-3733 (2021). R2: Huang, L., Liang, F., Feng, Y. X., Zhao, P. & Wang, W. H. On-chip integrated optical stretching and electrorotation enabling single-cell biophysical analysis. Microsyst Nanoeng 6 (2020). R3: Oh, M. J., Kuhr, F., Byfield, F. & Levitan, I. Micropipette Aspiration of Substrate-attached Cells to Estimate Cell Stiffness. Jove-J Vis Exp (2012). R4: Masaeli, M. et al. Multiparameter mechanical and morphometric screening of cells. Sci Rep-Uk 6 (2016). R5: Piergiovanni, M. et al. Deformation of leukaemia cell lines in hyperbolic microchannels: investigating the role of shear and extensional components. Lab Chip 20, 2539-2548 (2020). R6: Lee, L. M., Lee, J. W., Chase, D., Gebrezgiabhier, D. & Liu, A. P. Development of an advanced microfluidic micropipette aspiration device for single cell mechanics studies. Biomicrofluidics 10 (2016). R7: Lange, J. R. et al. Microconstriction arrays for high-throughput quantitative measurements of cell mechanical properties. Biophysical journal 109, 26-34 (2015); Ahmmed, S. M. et al. Multi-sample deformability cytometry of cancer cells. Apl Bioeng 2 (2018); and Apichitsopa, N., Jaffe, A. & Voldman, J. Multiparameter cell-tracking intrinsic cytometry for single-cell characterization. Lab Chip 18, 1430-1439 (2018).

As can be seen, the systems according to one or more embodiments achieve high-throughput and automatic sample processing and data analysis with low expense and simple fabricating processes. The microfluidic chips according to one or more embodiments include parallelized short microconstrictions to allow high-throughput induction of cells' deformation. The computational framework integrates cutting-edge technologies for object detection (such as YOLOv5), tracking (such as Deep SORT), and segmentation (ResUNet++), thereby being capable for capturing multiple fast-moving objects (such as cells) simultaneously in one FOV. Moreover, the computational framework can also be conjugated with biomechanical analysis based on cells' trajectories to obtain a quantification of cells' stiffness (such as c1 index) that is proportional to cells' elastic modulus.

Validation on cancer cell lines MCF-7 and MDA-MB-231, as an example for illustrative purpose, serves as a simulation of applying the system according to one or more embodiments to evaluate cancer metastasis. According to the results, both passage time and deformation index measured by the system vary significantly for the two cell lines. MDA-MB-231 cells are more invasive than MCF-7 cells (with lower passage time and higher deformation index), which is in harmony with the physiological difference between the two cell lines. The accuracy of classifying the two cell types based on optimal thresholds of passage time and deformation index achieves 87.8% and 67.7%, respectively. To demonstrate the efficiency of the system in distinguishing cancer cells from white blood cells, the efficiency of gating is tested using cell size calculated by the system, and achieves a high classifying accuracy of 89.5%. In order to evaluate the ability of the system in identifying heterogencities in metastatic potentials of cancer cells from the same tumor sample, the detecting assay with mixed groups containing MCF-7 and MDA-MB-231 in different ratios is run, which demonstrates that shifts in cancer cell populations are detectable by the system. These results demonstrate the system's great potential in clinical applications, including evaluating metastatic potentials for samples with heterogeneous phenotypes of cancer cells.

As used herein, the term “parallelized microconstrictions” or the like means the microconstrictions are configured in such a way that cells can flow into different microconstrictions separately and independently without interaction between cells in different microconstrictions. The term “parallelized” should not be understood as requiring all the side walls of the microconstrictions are strictly in parallel with each other.

Unless otherwise defined, the technical and scientific terms used herein have the plain meanings as commonly understood by those skill in the art to which the example embodiments pertain. Embodiments are illustrated in non-limiting examples. Based on the above disclosed embodiments, various modifications that can be conceived of by those skilled in the art fall within scope of the example embodiments.

What is claimed is:

1. A microfluidic chip for implementing cell deformability, comprising:
an inlet configured to receive cells;
an outlet configured to output the cells that have entered the microfluidic chip via the inlet;
a plurality of main channels disposed between the inlet and the outlet and provided with microconstrictions that are parallelized such that images of the cells are captured within a single field of view (FOV) when the cells pass through the microconstrictions and are deformed therein; and
one or more bypass channels disposed between the inlet and the outlet and independent from the plurality of main channels, thereby stabilizing pressure drop between the inlet and the outlet.

2. The microfluidic chip of claim 1, wherein the microconstrictions are categorized into m groups and the number of microconstrictions in each group is n, m and n being positive integers and greater than one, wherein the plurality of main channels are branched into m sub-channels, and each group of the microconstrictions is disposed in a separate respective sub-channel, and wherein the output of the sub-channels converge to a sink connecting to the outlet.

3. The microfluidic chip of claim 1, wherein each of the microconstrictions has a width in a range from 9 μm to 11 μm, a height in a range from 25 μm to 32 μm, and a length in a range from 55 μm to 75 μm, wherein the width is measured in a first direction, the height is measured in a second direction perpendicular to the first direction, and the length is measured in a third direction perpendicular to both the first direction and the second direction, and wherein the third direction is in parallel with moving direction of the cells within the microconstrictions.

4. The microfluidic chip of claim 1, wherein the one or more bypass channels consist of two bypass channels that surround the plurality of main channels.

5. The microfluidic chip of claim 1, wherein the plurality of main channels consist of two main channels, and the microconstrictions consist of four groups of microconstrictions,
wherein each main channel is branched into two sub-channels before reaching respective group of microconstrictions such that each group of the microconstrictions is disposed within respective sub-channel, and the end of the microconstrictions facing the outlet are connected to a sink connecting to the outlet,
wherein the one or more bypass channels consist of two bypass channels that surround the main channels, the sub-channels, the microconstrictions, and the sink.

6. A system for determining cell deformability, comprising:
a microfluidic chip that includes an inlet, an outlet, a plurality of main channels disposed between the inlet and the outlet and provided with parallelized microconstrictions for deforming cells, and one or more bypass channels that are independent from the main channels and adjust pressure drop between the inlet and the outlet;
delivering means configured to deliver the cells to the microfluidic chip via the inlet such that the cells are deformed by the parallelized microconstrictions;
an image capturing device configured to collect data of the cells when the cells travel through the parallelized microconstrictions to generate collected data, the collected data being related to morphological and motional parameters of the cells; and
a computing device communicating with the image capturing device and including a computational framework that is implemented with an artificial neural network (ANN) and is configured to determine the cell deformability based on the collected data received from the image capturing device.

7. The system of claim 6, wherein the delivering means includes a syringe pump that pumps a fluid sample including the cells into the inlet of the microfluidic chip such that the cells flow through the plurality of main channels under pressure difference between the inlet and the outlet and are deformed when travelling through the parallelized microconstrictions.

8. The system of claim 6, wherein the computational framework is configured to automate generation of a training set by using background subtraction method for training the ANN.

9. The system of claim 8, wherein the ANN includes a cell detector for detecting positions of the cells, the cell detector being selected from a group consisting of YOLOv5, YOLOv6, and YOLOv7.

10. The system of claim 9, wherein the ANN includes a cell tracker for tracking trajectory of the cells, the cell tracker being selected from a group consisting of Deep SORT and Strong SORT, wherein the cell detector and the cell tracker determine passage time for each of the cells that have passed through the microconstrictions.

11. The system of claim 10, wherein the ANN is configured to set focusing areas for determining entry time and leaving time of the cells when traveling through the microconstrictions.

12. The system of claim 10, wherein the ANN includes a segmentation model for determining deformation index and size for each of the cells that have passed through the microconstrictions.

13. The system of claim 12, wherein the segmentation model is ResUnet++, and the deformation index is defined by (H−W)/(H+W), H being the length of a cell when the cell is within a microconstriction, W being the width of the cell when the cell is within the microconstriction.

14. A method for determining cell deformability, comprising:
delivering a fluid sample including cells into a microfluidic chip such that the cells flow through a plurality of main channels of the microfluidic chip and are deformed by a plurality of parallelized microconstrictions;
recording images of the cells within a single field of view (FOV) by a camera when the cells travel through the parallelized microconstrictions, thereby generating recorded images; and
determining deformation index for the cells by processing the recorded images by using a trained artificial neural network (ANN).

15. The method of claim 14, further comprising:
automating generation of a training set by using a background subtraction method, and
training an ANN using the training set, thereby producing the trained ANN.

16. The method of claim 15, wherein training the ANN includes:
training YOLOv5 using images and annotation derived from the recorded images by using the background subtraction method;
tracking the cells using YOLOv5 and Deep SORT;
cutting the images derived from the recorded images when the cells crossing focusing areas to obtain cut images; and
training ResUnet++ using the cut images.

17. The method of claim 14, further comprising:
detecting the cells from the recorded images by using trained YOLOv5;
tracking trajectory of the cells by using Deep SORT; and
calculating deformation index and size of the cells by using ResUnet++.

18. The method of claim 17, further comprising:
setting focusing areas for determining entry time and leaving time for each of the cells when traveling through respective microconstriction; and
calculating passage time for each of the cells by using the trained YOLOv5 and Deep SORT.

19. The method of claim 18, wherein calculating deformation index and size of the cells includes:
calculating the deformation index for each cell by calculating (H−W)/(H+W), H being the length of a cell when the cell is in respective microconstriction, W being the width of the cell when the cell is in the respective microconstriction; and
calculating the size of each cell by using the following equation:
$${t}_{creep} = {c}_{1} \cdot {( 1 - \frac{{c}_{2}}{{A}_{cell}^{\frac{1}{2}}} )}^{\frac{1}{β}}$$
wherein t_{creep} is creep time,
$${c}_{1} = {E}_{cell}^{\frac{1}{β}} / Δ {\overline{P}}^{\frac{1}{β}} ,$$
E_{cell} is Young's modulus of cell, β is a power-law exponent, ΔP̅ is mean pressure drop across a respective microconstriction, c₂=Rₑ·\sqrt{π}, and Rₑ is half of width of the respective microconstriction, and wherein A_{cell} is size of the cell.

20. The method of claim 19, further comprising categorizing different cell types based on passage time and size of the cells by using a support vector machine algorithm.
